SEQUENCE LISTING (21 sequences)

SEQ ID NO: 1 - protein, 408 aa, Methanosarcina thermophila
MKVLVINAGSSSLKYQLIDMTNESALAVGLCERIGIDNSIITQKKFDGKKLEKLTDLPTHKDALEEVVKALTDDEFGVIKDMGEINAVGHRVVHGGEKFTTSALYDEGVEKAIKDCFELAPLHNPPNMMGISACAEIMPGTPMVIVFDTAFHQTMPPYAYMYALPYDLYEKHGVRKYGFHGTSHKYVAERAALMLGKPAEETKIITCHLGNGSSITAVEGGKSVETSMGFTPLEGLAMGTRCGSIDPAIVPFLMEKEGLTTREIDTLMNKKSGVLGVSGLSNDFRDLDEAASKGNRKAELALEIFAYKVKKFIGEYSAVLNGADAVVFTAGIGENSASIRKRILTGLDGIGIKIDDEKNKIRGQEIDISTPDAKVRVFVIPTNEELAIARETKEIVETEVKLRSSIPV

SEQ ID NO: 2 - protein, 587 aa, Geobacillus stearothermophilus
MKRKTKIVCTIGPASESVDKLVQLMEAGMNVARLNFSHGDHEEHGRRIANIREAAKRTGRTVAILLDTKGPEIRTHNMENGAIELKEGSKLVISMSEVLGTPEKISVTYPSLIDDVSVGAKILLDDGLISLEVNAVDKQAGEIVTTVLNGGVLKNKKGVNVPGVKVNLPGITEKDRADILFGIRQGIDFIAASFVRRASDVLEIRELLEAHDALHIQIIAKIENEEGVANIDEILEAADGLMVARGDLGVEIPAEEVPLIQKLLIKKCNMLGKPVITATQMLDSMQRNPRPTRAEASDVANAIFDGTDAVMLSGETAAGQYPVEAVKTMHQIALRTEQALEHRDILSQRTKESQTTITDAIGQSVAHTALNLDVAAIVTPTVSGKTPQMVAKYRPKAPIIAVTSNEAVSRRLALVWGVYTKEAPHVNTTDEMLDVAVDAAVRSGLVKHGDLVVITAGVPVGETGSTNLMKVHVISDLLAKGQGIGRKSAFGKAVVAKTAEEARQKMVDGGILVTVSTDADMMPAIEKAAAIITEEGGLTSHAAVVGLSLGIPVIVGVENATTLFKDGQEITVDGGFGAVYRGHASVL

SEQ ID NO: 3 - protein, 244 aa, Pyrococcus furiosus
GSSHHHHHHSSGLVPRGSHMRIVFDIGGSVLVPENPDIDFIKEIAYQLTKVSEDHEVAVVVGGGKLARKYIEVAEKFNSSETFKDFIGIQITRANAMLLIAALREKAYPVVVEDFWEAWKAVQLKKIPVMGGTHPGHTTDAVAALLAEFLKADLLVVITNVDGVYTADPKKDPTAKKIKKMKPEELLEIVGKGIEKAGSSSVIDPLAAKIIARSGIKTIVIGKEDAKDLFRVIKGDHNGTTIEP

SEQ ID NO: 4 - protein, 375 aa, Thermotoga maritima
MFRILTINPGSTSTKLSIFEDERMVKMQNFSHSPDELGRFQKILDQLEFREKIARQFVEETGYSLSSFSAFVSRGGLLDPIPGGVYLVDGLMIKTLKSGKNGEHASNLGAIIAHRFSSETGVPAYVVDPVVVDEMEDVARVSGHPNYQRKSIFHALNQKTVAKEVARMMNKRYEEMNLVVAHMGGGISIAAHRKGRVIDVNNALDGDGPFTPERSGTLPLTQLVDLCFSGKFTYEEMKKRIVGNGGLVAYLGTSDAREVVRRIKQGDEWAKRVYRAMAYQIAKWIGKMAAVLKGEVDFIVLTGGLAHEKEFLVPWITKRVSFIAPVLVFPGSNEEKALALSALRVLRGEEKPKNYSEESRRWRERYDSYLDGILR

SEQ ID NO: 5 - protein, 220 aa, Thermotoga maritima
MMAYLVFLGPPGAGKGTYAKRLQEITGIPHISTGDIFRDIVKKENDELGKKIKEIMERGELVPDELVNEVVKRRLSEKDCERGFILDGYPRTVAQAEFLDGFLKTQNKELTAAVLFEVPEEVVVQRLTARRICPKCGRIYNLISLPPKEDELCDDCKVKLVQREDDKEETVRHRYKVYLEKTQPVIDYYDKKGILKRVDGTIGIDNVIAEVLKIIGWSDK

SEQ ID NO: 6 - protein, 194 aa, Sulfolobus acidocaldarius
MKIGIVTGIPGVGKSTVLAKVKEILDNQGINNKIINYGDFMLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMK

SEQ ID NO: 7 - DNA, 981 bp, Artificial Sequence
  Adenylate kinase from Sulfolobus acidocaldarius with unstructured linker and VNAR sequence
atgaagatcggcattgtgaccggcattccgggcgttggcaaaagcaccgttctggcaaaggtgaaggagatcctggacaaccagggcattaataacaaaattattaattatggtgattttatgctggcgaccgcgctgaagctgggctacgcaaaagatcgtgacgaaatgcgcaaactgagcgtggaaaaacagaagaagctgcagattgatgcggcgaagggcattgcggaagaggcacgcgcgggcggcgaaggctacctgtttatcgatacccatgcggtgatccgcaccccgagcggttatctgccgggcctgccgtcttacgtgattacggaaatcaacccgagcgttatttttctgctggaggcagatccgaagattattctgagccgccagaagcgcgataccacccgcaaccgcaacgattatagcgacgaaagcgttatcctggagaccatcaactttgcgcgctatgcggcaaccgcgagcgcggttctggcaggctctaccgttaaagtgatcgtgaacgtggagggtgatccaagcatcgcggcgaacgaaatcattcgcagcatgaaacagtcgaccggtggtggtggttctggtggtggtggttctggtggtggtggttctggatccgcacgtgttgatcagacaccgcgtagcgttaccaaagaaaccggtgaaagcctgaccattaattgtgttctgcgtgatgcaagctatgcactgggtagcacctgttggtatcgtaaaaaaagcggcagcaccaatgaagaaagcattagcaaaggtggtcgttatgttgaaaccgtgaatagcggtagcaaaagctttagcctgcgtattaatgatctgaccgttgaagatggtgggacctaccgttgtggtctgggtgttgccggtggttattgtgattatgcactgtgcagcagccgttatgcagaatgtggtgatggtactgcagttaccgtgaat

SEQ ID NO: 8 - protein, 115 aa, Artificial Sequence
  VNAR sequence for hen egg lysozyme specific single domain antibody from shark
GSARVDQTPRSVTKETGESLTINCVLRDASYALGSTCWYRKKSGSTNEESISKGGRYVETVNSGSKSFSLRINDLTVEDGGTYRCGLGVAGGYCDYALCSSRYAECGDGTAVTVN

SEQ ID NO: 9 - protein, 287 aa, Artificial Sequence
  Fusion protein sequence for adenylate kinase from Sulfolobus acidocaldarius with unstructured linker and VNAR sequence
MLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMKQSTGGGGSGGGGSGGGGSGSARVDQTPRSVTKETGESLTINCVLRDASYALGSTCWYRKKSGSTNEESISKGGRYVETVNSGSKSFSLRINDLTVEDGGTYRCGLGVAGGYCDYALCSSRYAECGDGTAVTVN

SEQ ID NO: 10 - protein, 297 aa, Artificial Sequence
  Fusion protein sequence for adenylate kinase from Sulfolobus acidocaldarius with helical linker and VNAR sequence
MLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMKQSTLAEAAAKEAAAKEAAAKEAAAKAAAGSARVDQTPRSVTKETGESLTINCVLRDASYALGSTCWYRKKSGSTNEESISKGGRYVETVNSGSKSFSLRINDLTVEDGGTYRCGLGVAGGYCDYALCSSRYAECGDGTAVTVN

SEQ ID NO: 11 - protein, 299 aa, Artificial Sequence
  Fusion protein sequence for VNAR sequence with helical linker and adenylate kinase from Sulfolobus acidocaldarius
MGSARVDQTPRSVTKETGESLTINCVLRDASYALGSTCWYRKKSGSTNEESISKGGRYVETVNSGSKSFSLRINDLTVEDGGTYRCGLGVAGGYCDYALCSSRYAECGDGTAVTVNSTLAEAAAKEAAAKEAAAKEAAAKAAAGSMLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMK

SEQ ID NO: 12 - protein, 289 aa, Artificial Sequence
  Fusion protein sequence for VNAR sequence with unstructured linker and adenylate kinase from Sulfolobus acidocaldarius
MGSARVDQTPRSVTKETGESLTINCVLRDASYALGSTCWYRKKSGSTNEESISKGGRYVETVNSGSKSFSLRINDLTVEDGGTYRCGLGVAGGYCDYALCSSRYAECGDGTAVTVNSTGGGGSGGGGSGGGGSGSMLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMK

SEQ ID NO: 13 - protein, 128 aa, Artificial Sequence
  VHH sequence for hen egg lysozyme specific single domain antibody from camelid
EVQLQASGGGSVQAGQSLRLSCATSGATSSSNCMGWFRQAPGKEREGVAVIDTGRGNTAYADSVQGRLTISLDNAKNTLYLQMNSLKPEDTAMYYCAADTSTWYRGYCGTNPNYFSYWGQGTQVTVSS

SEQ ID NO: 14 - protein, 302 aa, Artificial Sequence
  Fusion protein sequence for adenylate kinase from Sulfolobus acidocaldarius with unstructured linker and VHH sequence
MLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMKQSTGGGGSGGGGSGGGGSGSEVQLQASGGGSVQAGQSLRLSCATSGATSSSNCMGWFRQAPGKEREGVAVIDTGRGNTAYADSVQGRLTISLDNAKNTLYLQMNSLKPEDTAMYYCAADTSTWYRGYCGTNPNYFSYWGQGTQVTVSS

SEQ ID NO: 15 - protein, 312 aa, Artificial Sequence
  Fusion protein sequence for adenylate kinase from Sulfolobus acidocaldarius with helical linker and VHH sequence
MLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMKQSTLAEAAAKEAAAKEAAAKEAAAKAAAGSEVQLQASGGGSVQAGQSLRLSCATSGATSSSNCMGWFRQAPGKEREGVAVIDTGRGNTAYADSVQGRLTISLDNAKNTLYLQMNSLKPEDTAMYYCAADTSTWYRGYCGTNPNYFSYWGQGTQVTVSS

SEQ ID NO: 16 - protein, 312 aa, Artificial Sequence
  Fusion protein sequence for VHH sequence with helical linker and adenylate kinase from Sulfolobus acidocaldarius
MEVQLQASGGGSVQAGQSLRLSCATSGATSSSNCMGWFRQAPGKEREGVAVIDTGRGNTAYADSVQGRLTISLDNAKNTLYLQMNSLKPEDTAMYYCAADTSTWYRGYCGTNPNYFSYWGQGTQVTVSSSTLAEAAAKEAAAKEAAAKEAAAKAAAGSMLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMK

SEQ ID NO: 17 - protein, 301 aa, Artificial Sequence
  Fusion protein sequence for VHH sequence with unstructured linker and adenylate kinase from Sulfolobus acidocaldarius
EVQLQASGGGSVQAGQSLRLSCATSGATSSSNCMGWFRQAPGKEREGVAVIDTGRGNTAYADSVQGRLTISLDNAKNTLYLQMNSLKPEDTAMYYCAADTSTWYRGYCGTNPNYFSYWGQGTQVTVSSSTGGGGSGGGGSGGGGSGSMLATALKLGYAKDRDEMRKLSVEKQKKLQIDAAKGIAEEARAGGEGYLFIDTHAVIRTPSGYLPGLPSYVITEINPSVIFLLEADPKIILSRQKRDTTRNRNDYSDESVILETINFARYAATASAVLAGSTVKVIVNVEGDPSIAANEIIRSMK

SEQ ID NO: 18 - protein, 25 aa, Artificial Sequence
  Helical linker
LAEAAAKEAAAKEAAAKEAAAKAAA

SEQ ID NO: 19 - protein, 403 aa, Thermotoga maritima
MRVLVINSGSSSIKYQLIEMEGEKVLCKGIAERIGIEGSRLVHRVGDEKHVIERELPDHEEALKLILNTLVDEKLGVIKDLKEIDAVGHRVVHGGERFKESVLVDEEVLKAIEEVSPLAPLHNPANLMGIKAAMKLLPGVPNVAVFDTAFHQTIPQKAYLYAIPYEYYEKYKIRRYGFHGTSHRYVSKRAAEILGKKLEELKIITCHIGNGASVAAVKYGKCVDTSMGFTPLEGLVMGTRSGDLDPAIPFFIMEKEGISPQEMYDILNKKSGVYGLSKGFSSDMRDIEEAALKGDEWCKLVLEIYDYRIAKYIGAYAAAMNGVDAIVFTAGVGENSPITREDVCSYLEFLGVKLDKQKNEETIRGKEGIISTPDSRVKVLVVPTNEELMIARDTKEIVEKIGR

SEQ ID NO: 20 - protein, 231 aa, Thermotoga maritima
MRVLVKLSGEALSGEGGRGFDPERVNYIVNEIKSAIEEGFKIGIVVGAGNLFRGVELKNLTMTRADQIGLLGTVMNSVYLKDIFERSGLKARIYSQIVNLPDVERVNYDSIESALRENSILIFAGGTSNPFFTTDTAAVLRAQEMRAKLVVKATKVDGVYDKDPKKFPDAKKIPHLTFSEAMKMGLKVMDAEAFALCKKLGITVKVINFFEPGTLLKALKGEDVGSTVVPD

SEQ ID NO: 21 - protein, 227 aa, Sulfolobus acidocaldarius
MKLTLKVSGKFFDEENSENLSLLRDVIIDLVNNGHRVAVVTGGGGTARRYISMGRKLNLNESHLDILGILVSRLNAQLLLFSLDNIAYPKVPESIEDFNERWASGKVVITGGFQPGQSTAGVAALVSEIINADYLVLATNVNGVYTKDPQKFVDAKLLPKLTVSELKTILEGSQSVNAGKYELLDPLAIKIVERSKIKVLVINFKDLNKLPNILKGNEILGSVVVPE

(19) United States
(12) Patent Application Publication
(10) Pub. No.: US 2014/0315215 A1
(43) Pub. Date: Oct. 23, 2014

(54) THERMOSTABLE ASSAY REAGENTS

(71) Applicants: John Mark Sutton, Salisbury Wiltshire (GB); Philip James Alister Skipper, Lincolnshire (GB)

(72) Inventors: John Mark Sutton, Salisbury Wiltshire (GB); Philip James Alister Skipper, Lincolnshire (GB)

(73) Assignee: The Secretary of State for Health, London (GB)

(21) Appl. No.: 14/344,771

(22) PCT Filed: Sep. 14, 2012
(86) PCT No.: PCT/GB2012/052281

§ 371 Date: Jul. 7, 2014

(30) Foreign Application Priority Data
Sep. 14, 2011 (GB) 1115911.8

(51) Int. Cl. G01N 33/573; C07K 16/44; C12N 9/12

(52) U.S. Cl. CPC G01N 33/573; C12N 9/12; C07K 16/44; C07K 2319/30
USPC 435/7.4; 435/188

(57) ABSTRACT

There is provided a single-chain fusion protein comprising: (i) a thermostable kinase and (ii) a single-domain antibody or single-domain antibody fragment. There is also provided a method of preparing a single-domain antibody or single-domain antibody fragment, the method comprising: (i) expressing the single-domain antibody or antibody fragment as a single-chain fusion protein with a thermostable kinase, in a host cell such as E. coli; and (ii) purifying the fusion protein from the cytoplasm of the host cell.

The invention relates to the expression and production of highly stable assay reagents comprising single-domain antibodies or antibody fragments, and uses thereof for detecting an analyte on or within a sample.

Unlike common antibodies, which are composed of two heavy chains and two light chains, a single-domain antibody (sdAb) is a single-chain antibody polypeptide consisting of a single monomeric variable antibody domain.

Single-domain antibodies are typically about 110 amino acids long and have a typical molecular weight in the region of 12-15 kDa. As such, single-domain antibodies are much smaller than common antibodies (150-160 kDa), and even smaller than Fab fragments (which consist of one light chain and half a heavy chain and have a molecular weight of about 50 kDa) and single-chain variable fragments (which consist of two variable domains, one from a light and one from a heavy chain, and have a molecular weight of about 25 kDa).

The first single-domain antibodies, called “V_{H}H fragments”, were engineered from heavy-chain antibodies found in camelids. Single-domain antibodies called “V_{NAR} fragments” can also be obtained from the heavy-chain antibodies of cartilaginous fishes (Ig_{NAR}, ‘immunoglobulin new antigen receptor’). Single-domain antibodies can also be obtained by splitting the dimeric variable domains from common immunoglobulin G (IgG) (eg. from humans or mice) into monomers. Although most research into single-domain antibodies is currently based on heavy-chain variable domains, single-domain antibodies derived from light chains have also been shown to bind specifically to target epitopes.

Single-domain antibodies have been obtained in the art by immunization of dromedaries, camels, llamas, alpacas or sharks with the desired antigen and subsequent isolation of the mRNA coding for heavy-chain antibodies. A gene library of single-domain antibodies containing several million clones is produced by reverse transcription and PCR, and screening techniques such as phage display and ribosome display are used to identify clones binding the antigen.

Alternatively, single-domain antibodies have been made in the art from common murine or human IgGs. A problem with this approach is that the binding region of a common IgG consists of two domains (V_{H} and V_{L}), which tend to dimerize or aggregate because of their lipophilicity. Monomerization is usually accomplished by replacing lipophilic by hydrophilic amino acids, but often results in a loss of affinity to the antigen.

Given their simple domain architecture, single-domain antibodies lend themselves to recombinant expression as fusion proteins.

The ease of genetic manipulation of E. coli makes it an attractive host for expressing antibodies. It is known in the art to express labelled antibody fragments from the periplasm of E. coli. By way of example, Dooley M. F. et al., Mol. Imm., (2003) 40:25-33; Arbabi M. et al, FEBS Letters (1997) 414:521-526; and Nuttall S. D. et al., Eur. J. Biochem. 2003 Sep. 270(17):3543-54 describe high level expression of camelid and shark antibodies in the E. coli periplasm. However, these expression systems are associated with a number of technical problems, such as unreliable yields and inconsistent secretion of active antibody polypeptides.

Low-level cytoplasmic expression of antibodies has also been described (see Bossi S. et al., Protein Expression and Purification, Volume 72, Issue 1, July 2010, pages 55-58), using specialized strains of E. coli with altered conditions and in the presence of stabilising antigen. The technical challenges associated with cytoplasmic expression of antibodies reflect the requirement for the formation of one or more intra-chain disulfide bonds within the single domain antibody peptide chain, which are essential to constrain the structure of the antibody, hence yielding high affinity binding properties. The proteins involved in forming disulfide bonds and in shuffling these within complex proteins to generate a single preferred conformation are situated within the periplasm of E. coli. In contrast, the cytoplasm of E. coli is generally regarded as being a reductive environment, which does not favour the formation of disulfide bonds and actively promotes their reduction to two free sulfhydryl groups. A small number of E. coli strains have been engineered to provide periplasm-like conditions in the cytoplasm—eg. by generating a non-reducing environment (eg. by knocking out cytoplasmic thioredoxin). However, these strains tend to grow less well than native strains, leading to a reduction in the biomass generated and also give lower expression levels, hence reducing the yield of protein.

In view of these technical problems, cytoplasmically expressed antibody fragments or domains thereof are almost universally mis-folded and unable to bind to their target antigen with high affinity.

There is therefore a need in the art for reliable and efficient means to express single-domain antibodies and antibody fragments in the E. coli cytoplasm that overcome one or more of the problems encountered in the prior art.

The present invention meets this need in the art by expressing single-domain antibodies or antibody fragments in the E. coli cytoplasm as a fusion protein with a thermostable kinase.

Thus, in a first aspect of the invention, there is provided a single-chain fusion protein comprising: (i) a thermostable kinase; and (ii) a single-domain antibody or single-domain antibody fragment.

The invention also provides a method of preparing a single-domain antibody or antibody fragment, the method comprising: (i) expressing the single-domain antibody or antibody fragment as a single-chain fusion protein with a thermostable kinase, in a host cell such as E. coli; and (ii) purifying the fusion protein from the cytoplasm of the host cell.

It is known in the art to express and purify thermostable kinases as recombinant proteins from the cytoplasm of E. coli, providing more accessible and reproducible material than when isolated from the host thermophilic organisms (see WO 2005/093085, WO 2009/104013 and WO 2010/079357, incorporated herein by reference). As thermostable adenylate kinases (tAKs), such as those from Sulfolobus acidocaldarius and Thermotoga maritima, lack intramolecular disulfide bonds, these enzymes have been shown to fold correctly within the relatively oxidising environment of the E. coli cytoplasm.

Thermostable proteins have become increasingly used as fusion tags. In this regard, fusion of proteins which are intrinsically soluble even at high temperatures is known to promote folding of passenger proteins [de Marco et al., J. Biotech., (2004) 107:125-133; Fox J. D. et al., FEBS Lett. 2003 Feb. 27; 537(1-3):53-7; Kondo N. et al., Anal Biochem. 2009 Feb. 15; 385(2):278-85. Epub 2008 Nov. 19; Luke J. M. et al., J. Biotechnol. 2011 Feb. 10; 151(3):242-50. Epub 2010 Dec. 17.] This approach has not previously been used for the expression of recombinant antibodies, due to problems with obtaining the correct disulfide bond formation and the need for separate fusion tags on each antibody chain, mainly due to the cytoplasmic localisation of these fusion partners.

The present inventors have unexpectedly identified that, when expressed from the cytoplasm, the thermostable kinase component of the fusion protein actively promotes the folding of the single-domain antibody component and provides a stabilizing function, whilst retaining measurable kinase activity.

Furthermore, the fusion proteins of the invention can be purified from the cytoplasm of E. coli in active form with yields higher than would be expected for the same single-domain antibody or antibody fragment in the periplasm.

The conference of high-level thermal stability onto a single-domain antibody fragment has not been described previously. In this context “high level thermal stability” refers to thermal stability above 80° C., preferably above 90° C.

The high stability of the expressed fusion proteins advantageously allows their purification via a rapid protocol involving high temperature treatment of E. coli cell lysate, which is clarified and applied to a single chromatography column. The high stability of the expressed fusion proteins also advantageously allows for their storage at room temperature (rather than at 4° C. or below).

The high stability of the fusion protein of the invention is also advantageous if the fusion protein is used as a reporter molecule in detection assays. By way of example, the use of a highly stable fusion protein of the invention allows the addition of high levels of urea to the sample extractant or diluents, enabling improved antigen extraction and background reduction (as detailed in WO2010/079357) with no loss of detection sensitivity. Treatment of the sample with urea also allows the detection of different types of antigen, focussing on conserved epitopes which would not normally be solvent exposed.

As used herein, the term “single-domain antibody” embraces any single-chain antibody polypeptide consisting of a single monomeric variable antibody domain. Examples of single-domain antibodies include “V_{H}H fragments” (eg. engineered from camelid heavy-chain antibodies) and “V_{NAR} fragments” (eg. obtained from an Ig_{NAR} heavy-chain antibody of cartilaginous fishes, such as shark).

In one embodiment, the single-domain antibody or antibody fragment is a V_{NAR} fragment of a shark immunoglobulin new antigen receptor (Ig_{NAR}). One example of a V_{NAR} sequence of a single-domain antibody from shark is provided herein as SEQ ID NO: 8.

In one embodiment, the single-domain antibody or antibody fragment is a V_{H}H fragment of a camelid heavy-chain antibody. One example of a V_{H}H sequence of a single-domain antibody from camelid is provided herein as SEQ ID NO: 13.

The single-domain antibody component of the fusion protein binds an antigen.

As used herein, the term “single-domain antibody fragment” embraces a fragment of any “single-chain antibody” as defined herein. Examples of fragments include truncated genes that encode only the complementarity determining region (CDR), responsible for determining the antigen specificity; in the case of V_{NAR} fragments only CDR3, possibly constrained artificially by one or more conventional techniques known to those familiar in the art. Antibody fragments of the invention may be produced by recombinant DNA techniques. In one embodiment, the single-domain antibody fragment is at least 50 amino acids long, such as at least 60, 70, 80, 90 or 100 amino acids long.

The single-domain antibody fragment component of the fusion protein binds an antigen. A fragment of a single-domain antibody is capable of binding to the same antigen to which the full-length single-domain antibody binds.

Binding of the single-domain antibody or single-domain antibody fragment to the antigen (to form an antibody-antigen complex) can be detected using any conventional assay known in the art, and may form the basis of an assay for detecting an analyte comprising the antigen.

There are a number of technical advantages associated with the use of single-domain antibodies (or fragments thereof), as compared with conventional ‘whole antibodies’. By way of example, the comparatively low molecular mass of single-domain antibodies leads to good permeability in tissues, and a short plasma half-life. Furthermore, as they lack an Fc region, single-domain antibodies do not induce complement-triggered cytotoxicity. Some single-domain antibodies and antibody fragments bind to ‘hidden’ antigens (eg. the active sites of enzymes) that are not accessible to whole antibodies; this property has been shown to result from their extended CDR3 loop, which is able to penetrate such sites.

Single-domain antibodies and antibody fragments thereof therefore lend themselves to the development of novel diagnostic reagents and therapeutics.

In one embodiment, demonstrated in the Examples and Figures, an exemplary single-domain antibody is derived from either camelid heavy-chain antibody (V_{H}H) or the variable domain of shark Ig new antigen receptor (V_{NAR}).

As used herein, the term “antibody component” refers to the single-domain antibody and single-domain antibody fragment as defined herein.

Any kinase enzyme may be used in the present invention, so long as it is “thermostable”.

The term “thermostable kinase” refers to a kinase that retains kinase activity after exposure to heat—ie. that is relatively unaffected by high temperatures. In one embodiment of the invention, the kinase activity of a thermostable kinase after exposure to a temperature of between 50-120° C. is at least 70% (or at least 80%, 90%, 95% or 100%) of the kinase activity of the kinase prior to the exposure. In one embodiment, after exposure to 40° C. for 30 minutes, or after exposure to 50° C. for 30 minutes, or after exposure to 60° C. for 30 minutes, or after exposure to 70° C. for 30 minutes, or after exposure to 80° C. for 20 minutes, or after exposure to 90° C. for 10 minutes, or after exposure to 120° C. for 3 minutes, a thermostable kinase may retain at least 70% of the pre-exposure kinase activity (or at least 80%, 90%, 95% or 100% of the pre-exposure kinase activity).

Thermostable kinases may also be more resistant than non-thermostable kinases to a range of other biochemical and physical processes that routinely damage or destroy proteins or render them inactive, such as exposure to certain chemicals eg. chaotropes, free-radical damage, detergents, extremes of pH, exposure to proteases, protein cross-linking, encapsulation within non-permeable or semi-permeable membranes or polymers, or irreversible immobilisation onto surfaces (see for example: Daniel R. M. et al., Biochem J. 1982 207:641-4; Rees D C and Robertson A D, Protein Sci. 2001 10:1187-94; Burdette D S et al., Enzyme Microb Technol. 2000 27:11-18; Scandurra R. et al., Biochimie. 1998 November; 80(11):933-41; and Liao H H. Enzyme Microb Technol. 1993 April; 15(4):286-92). In one embodiment, after exposure to one or more of the biochemical and physical processes described above, thermostable kinases may retain at least 70% (or 80%, 90%, 95% or 100%) of their pre-exposure kinase activity.

It is a matter of routine for a skilled person to identify whether a kinase is “thermostable”, by measuring the “retained kinase activity” using any of the conventional tests available in the prior art.

A skilled person is familiar with a wide range of different thermostable kinases suitable for use in the present invention.

Thermostable kinases have a variety of recognized tertiary structures, and can be broadly classified into two groups based on their molecular architecture—ie. “multimeric”/“polymeric” kinases or “monomeric” kinases.

In one embodiment, the thermostable kinase forms multimers—ie. the thermostable kinase is a “multimeric” kinase, such a dimeric, trimeric, tetrameric, pentameric, hexameric, heptameric or octameric kinase. Multimeric (eg. dimeric, trimeric, tetrameric, hexameric, octameric) tertiary structures may be associated with an improved stability of the kinase to conditions such as temperature, pH, chemical denaturants, or proteases.

In one embodiment, the thermostable kinase forms trimers—ie. the thermostable kinase is a “trimeric” thermostable kinase.

By way of example, the thermostable kinase enzymes from Sulfolobus species (eg. S. acidocaldarius) have a trimeric structure with a central hydrophobic core that is the principle determinant in maintaining their activity at high temperatures. An example of a trimeric thermostable kinase is provided in SEQ ID NO: 6 (adenylate kinase from S. acidocaldarius).

In one embodiment, the thermostable kinase is a dimeric thermostable kinase. Acetate kinases, such as the enzyme from Methanosarcina thermophila (SEQ ID NO: 1 provided herein) and Thermotoga maritima (SEQ ID NO: 19), are dimeric.

In one embodiment, the thermostable kinase is a tetrameric thermostable kinase. Pyruvate kinases, such as the enzyme from Geobacillus stearothermophilus (SEQ ID NO: 2 provided herein), are tetrameric.

In one embodiment, the thermostable kinase is a hexameric thermostable kinase. Uridine monophosphate (UMP) kinases, such as the enzyme from Pyrococcus furiosus (SEQ ID NO: 3 provided herein), Sulfolobus acidocaldarius (SEQ ID NO: 21) and Thermotoga maritima (SEQ ID NO: 20), are hexameric.

In one embodiment, the thermostable kinase is an octameric thermostable kinase. Butyrate kinases, such as those from Thermotoga maritima (SEQ ID NO: 4 provided herein), are octameric.

Other thermostable kinase enzymes have a monomeric structure, exemplified by the adenylate kinases from Thermatoga species (eg. T. maritima, see SEQ ID NO: 5 provided herein). These kinases have a slightly longer polypeptide chain with an additional “lid” domain that affects the active site. Thus, in one embodiment, the thermostable kinase is a monomeric thermostable kinase.

In one embodiment, the thermostable kinase is an adenylate kinase, acetate kinase, UMP kinase, pyruvate kinase or butyrate kinase. In one embodiment, the thermostable kinase is a trimeric adenylate kinase. In one embodiment the thermostable kinase is a monomeric adenylate kinase. In one embodiment, the thermostable kinase is a dimeric acetate kinase. In one embodiment, the thermostable kinase is a hexameric UMP kinase.

In one embodiment, the thermostable kinase is a microbial kinase of an organism selected from the group consisting of Pyrococcus furiosus, P. abyssi, P. horikoshii, P. woesii; a Sulfolobus sp. such as Sulfolobus solfataricus, S. acidocaldarius, or S. shibatael; a Thermotoga sp. such as Thermatoga maritima or T. neapolitana; a Methanococcus spp.; Rhodothermus marinus, Thermococcus litoralis, Geobacillus stearothermophilus, and Methanosarcina thermophila.

In one embodiment, the thermostable kinase may be an A. fulgidus kinase, A. pernix kinase, A. pyrophilus kinase, B. caldotenax BT1 kinase, Bacillus species PS3 kinase, B. stearothermophilus 11057 kinase, B. stearothermophilus 12001 kinase, B. thermocatenulatus kinase, C. stercocorarium kinase, a G. stearothermophilus kinase, Methanococcus spp. kinase, M. thermophila kinase, M. ruber kinase, P. abyssi kinase, P. furiosus kinase, P. horikoshii kinase, P. woesii kinase, R. marinus kinase, S. acidocaldarius kinase, S. shibatae kinase, S. solfataricus kinase, T. ethanolicus kinase, T. thermosulfurogenes kinase, T. celere kinase, T. litoralis kinase, T. aquaticus YT1 kinase, T. caldophilus GK24 kinase, T. thermophilus HB8 kinase, T. maritima kinase, or a T. neapolitana kinase.

In one embodiment, the thermostable kinase is a T. maritima monomeric adenylate kinase. In one embodiment, the thermostable kinase is a T. maritima dimeric acetate kinase. In one embodiment, the thermostable kinase is a S. acidocaldarius trimeric adenylate kinase. In one embodiment, the thermostable kinase is a S. acidocaldarius hexameric UMP kinase. In one embodiment, the thermostable kinase is a T. maritima hexameric UMP kinase.

In one embodiment, the term “thermostable kinase” embraces sequence variants of any of the thermostable kinases of the invention discussed herein, and also embraces fragments thereof (including fragments of the sequence variants, and sequence variants of the fragments).

In this regard, the genetic modification of thermostable kinases has been shown to provide significant increases in thermal stability. A comparison of the thermostability of a range of kinase enzymes with the defined 3-D structure of the trimeric (archaeal) AKs has identified amino acids that influence stability (Vonrhein et al. (1998) J. Mol. Biol. 282:167-179 and Criswell et al. (2003) J. Mol. Biol. 330:1087-1099)

Genetically engineered variants of thermostable kinases showing modified (eg. improved) thermostability may be identified via the specific site-directed mutagenesis of amino acids (eg. amino acids believed to form part of the central core packing region of the trimeric molecule) or by random “directed evolution” methods where the whole kinase molecule is subjected to subsequent rounds of mutagenesis and selection/screening for molecules with improved properties.

In one embodiment, kinase activity of the “thermostable kinase” polypeptide is not required. In accordance with this embodiment, the sole purpose of the “thermostable kinase” component of the fusion protein is to facilitate (eg. increase) production of the single-domain antibody component. In accordance with this embodiment, the term “thermostable kinase” embraces sequence variants and truncated polypeptides that have a modified (eg. reduced) kinase activity as compared with the kinase activity of the reference kinase. By way of example, the variant or truncated polypeptide may have substantially no kinase activity.

In one embodiment, the thermostable kinase comprises or consists of an amino acid sequence having at least 70%, 80%, 85%, 90%, 95%, 99% or 100% sequence identity to the amino acid sequence of any of SEQ ID NOs: 1-6, or a fragment thereof comprising at least 50, 75, 100, 125, 150, 175, 200, 225, 250, 275, 300, 325, 350, 375, 400, 425, 450, 475, 500, 525 or 550 consecutive amino acids thereof.

In one embodiment, the thermostable kinase comprises or consists of an amino acid sequence comprising at least 50, 75, 100, 125, 150, 175, 200, 225, 250, 275, 300, 325, 350, 375, 400, 425, 450, 475, 500, 525 or 550 consecutive amino acids of any of SEQ ID NOs: 1-6, or an amino acid sequence having at least 70%, 80%, 85%, 90%, 95%, 99% or 100% sequence identity thereto.

Other examples of suitable reporter kinases are disclosed in WO 00/46357 and WO 2005/093085, which are hereby incorporated by reference in their entirety.

As used herein, the term “thermostable kinase component” refers to the thermostable kinase (including variants/fragments thereof) as defined herein.

The fusion protein of the invention comprises both (i) a thermostable kinase component; and (ii) a single-domain antibody or single-domain antibody fragment component.

The thermostable kinase and antibody components of the fusion protein can be fused in any order. Thus, in one embodiment, the thermostable kinase is expressed N-terminally to the single-domain antibody/antibody fragment in the fusion protein (ie. N-[thermostable kinase]-[antibody component]-C). In an alternative embodiment, the single-domain antibody/antibody fragment of the fusion protein is expressed N-terminally to the thermostable kinase in the fusion protein (ie. N-[antibody component]-[thermostable kinase]-C).

In one embodiment, the fusion protein of the invention comprises multiple (ie. at least 2 or more, such as 3, 4, 5, 6, 7 or 8) single-domain antibodies and/or antibody fragments, which may be the same or different.

By way of example, the fusion protein may comprise a thermostable kinase and two or more single-domain antibodies or antibody fragments, which may be the same, or may be different. For example, each thermostable kinase may be fused to multiple single-domain antibodies and/or antibody fragments, which may be the same or different. For example, the fusion protein may comprise a single-domain antibody or antibody fragment fused at the N-terminus of the thermostable kinase, and another single-domain antibody or antibody fragment fused at the C-terminus of the thermostable kinase, where the N-terminal single-domain antibody/antibody fragment and the C-terminal single-domain antibody/antibody fragment may be the same, or may be different.

As such, in one embodiment, the order of the components within the fusion protein is:
N-[antibody component 1]-[thermostable kinase]-[antibody component 2]-C (where antibody component 1 and 2 can be the same or different).

The different single-domain antibodies/antibody fragments within the fusion protein may differ from each other in terms of their binding activity. In this regard, the different single-domain antibodies or antibody fragments fused to the same thermostable kinase may bind to different analytes, or may bind to different epitopes of the same analyte, or may bind to the same analyte/epitope but with different affinities. In one embodiment, the different analytes bound by the single-domain antibodies or antibody fragments may be (or be derived from/components of) different biological infectious or non-infectious agents (eg. different bacterial or viral antigens). In one embodiment, the different analytes bound by the single-domain antibodies or antibody fragments may be a combination of (i) analytes from one or more different biological infectious/non-infectious agents and (ii) analytes having a detection function (“detector molecules”.

In one embodiment, the fusion protein of the invention comprises multiple (ie. at least 2 or more, such as 3, 4, 5, 6, 7 or 8) thermostable kinases, which may be the same or different.

By way of example, the fusion protein may comprise a single-domain antibody or antibody fragment and two or more thermostable kinases, which may be the same, or may be different. For example, each single-domain antibody or antibody fragment may be fused to multiple (eg. 2, 3, 4, 5, 6, 7 or 8) thermostable kinases, which may be the same or different. For example, the fusion protein may comprise a thermostable kinase fused at the N-terminus of the single-domain antibody or antibody fragment, and another thermostable kinase fused at the C-terminus of the single-domain antibody or antibody fragment, where the N-terminal thermostable kinase and the C-terminal thermostable kinase may be the same, or may be different.

As such, in one embodiment, the order of the components within the fusion protein is:
N-[thermostable kinase 1]-[antibody component]-[thermostable kinase 2]-C where thermostable kinase 1 and 2 can be the same or different.

The different thermostable kinases may differ from each other in terms of (for example) their target, and/or in terms of their level of kinase activity.

In one embodiment, the molecular weight of the single-domain antibody or antibody fragment component is over 50% of the molecular weight of the thermostable kinase (such as over 60%, 70%, 80% or 90% of the molecular weight of the thermostable kinase). In one embodiment, the total molecular weight of the single-domain antibody or antibody fragment component is greater than the molecular weight of the thermostable kinase. In one embodiment, the total molecular weight of the single-domain antibody or antibody fragment component forms over 50% of the molecular weight of the fusion protein (such as over 60%, 70%, 80% or 90% of the molecular weight of the fusion protein).

The fusion protein of the invention may be prepared using conventional techniques (eg. as described in Examples 1, 2, 6 and 7), such as by fusing a nucleic acid sequence encoding a thermostable kinase (as defined above) and a nucleic acid sequence encoding a single-domain antibody or antibody fragment (as defined above).

The present invention thus provides a polynucleotide sequence encoding a fusion protein of the invention as defined herein.

The nucleic acid sequences encoding the thermostable kinase and antibody components of the fusion protein can be arranged/fused in any order. Thus, in one embodiment, the nucleic acid sequence encoding the thermostable kinase may be located/fused 5′ to the nucleic acid sequence encoding the single-domain antibody or antibody fragment. Alternatively, the nucleic acid sequence encoding the single-domain antibody or antibody fragment may be located/fused 5′ to the nucleic acid sequence encoding the thermostable kinase.

In one embodiment, the polynucleotide sequence encoding the fusion protein of the invention comprises multiple (ie. at least 2 or more) nucleic acid sequences encoding single-domain antibodies and/or antibody fragments, which may be the same or different (eg. as discussed above).

Alternatively, or in addition, the polynucleotide sequence encoding the fusion protein of the invention may comprise multiple (ie. at least 2 or more) nucleic acid sequences encoding thermostable kinases, which may be the same or different (eg. as discussed above).

In one embodiment, the polynucleotide sequence encodes a fusion protein as discussed above, comprising a thermostable kinase and two single-domain antibodies or antibody fragments, which may be the same or different. In accordance with this embodiment, the polynucleotide sequence encoding the fusion protein comprises a nucleic acid sequence encoding a thermostable kinase and nucleic acid sequences encoding two single-domain antibodies or antibody fragments. For example, the polynucleotide sequence may comprise the following sequences in a 5′ to 3′ direction: a nucleic acid sequence that encodes a first single-domain antibody or antibody fragment, a nucleic acid sequence that encodes a thermostable kinase, and a nucleic acid sequence that encodes a second single-domain antibody or antibody fragment, wherein the first and second single-domain antibodies/fragments are the same or different.

In one embodiment, the polynucleotide sequence encodes a fusion protein as discussed above, comprising a single-domain antibody or antibody fragment and two thermostable kinases. In accordance with this embodiment, the polynucleotide sequence encoding the fusion protein comprises a nucleic acid sequence encoding a single-domain antibody or antibody fragment and nucleic acid sequences encoding two thermostable kinases. For example, the polynucleotide sequence may comprise the following sequences in a 5′ to 3′ direction: a nucleic acid sequence that encodes a first thermostable kinase, a nucleic acid sequence that encodes a single-domain antibody or antibody fragment, and a nucleic acid sequence that encodes a second thermostable kinase, wherein the first and second thermostable kinases are the same or different.

The polynucleotide sequence may optionally comprise one or more linker regions located between the coding sequences for the thermostable kinase and single-domain antibody components of the fusion protein.

As such, the polynucleotide sequence may comprise the following components in any of the following orders:

5′-[thermostable kinase coding sequence]-[linker]-[antibody coding sequence]-3′

5′-[antibody coding sequence]-[linker]-[thermostable kinase coding sequence]-3′

5′-[antibody coding sequence]-[linker]-[thermostable kinase coding sequence]-[linker]-[antibody coding sequence]-3′

5′-[thermostable kinase coding sequence]-[linker]-[antibody coding sequence]-[linker]-[thermostable kinase coding sequence]-3′

Examples of suitable conventional linker sequences known in the art include unstructured linkers (eg. (GGGGS)n where n=3-5) or helical linkers (eg. LAEAAAKEAAAKEAAAKEAAAKAAA).

The polynucleotide sequence may optionally comprise one or more sequences encoding a cleavage site, which may be located between the coding sequences for the thermostable kinase and single-domain antibody components of the fusion protein. The encoded cleavage site may be recognised by a protease. This allows separation of the thermostable kinase and the single-domain antibody or antibody fragment, if required, by cleavage at the cleavage site. For example, the thermostable kinase and single domain antibody components may be separated by a cleavage site recognised by factor Xa (IEGR↓), enterokinase (DDDDK↓), or TEV protease (ENLYFQ↓G). Other examples of cleavage sites are described in the literature.

Optionally the components of the fusion protein may be separated by the inclusion of an intein cleavage site between the thermostable kinase and the antibody or antibody fragment. This may require the additional presence of an intein domain at the C-terminus of the fusion or positioned sequentially to the thermostable kinase domain. Hence, in accordance with this embodiment, the fusion protein may take one of the following configurations:

N-Thermostable kinase-intein domain-intein cleavage site-Antibody component-C or

N-Thermostable kinase-intein cleavage site-Antibody component-intein domain-C

The present invention also provides a plasmid comprising a polynucleotide sequence encoding a fusion protein of the invention as defined herein.

The plasmid may comprise one or more additional polynucleotide sequences, which may be operably linked in frame to the polynucleotide sequence encoding the fusion protein. By way of example, the plasmid may comprise a polynucleotide sequence encoding a polypeptide tag or label, and expression from the plasmid may produce a tagged/labelled fusion protein. Optionally, any additional polynucleotide sequence may be separated from the polynucleotide sequence encoding the fusion protein by a linker sequence and/or by a sequence encoding a cleavage site, as discussed above. The presence of a cleavage site allows the fusion protein to be separated from any additional polypeptide (eg. tag or label) encoded by the additional polynucleotide sequence.

Gene expression can be driven from any of the described promoter systems familiar to those with knowledge in the art, such as IPTG-inducible lacZ based promoters or tet promoters. Expression may for example be driven by the malate dehydrogenase promoter (MDH) as described previously (Ungurs et al., J. Hospital Infection, Vol. 74, 2010, pages 144-151 “Quantitative measurement of the efficacy of protein removal by cleaning formulations; comparative evaluation of prion-directed cleaning chemistries”).

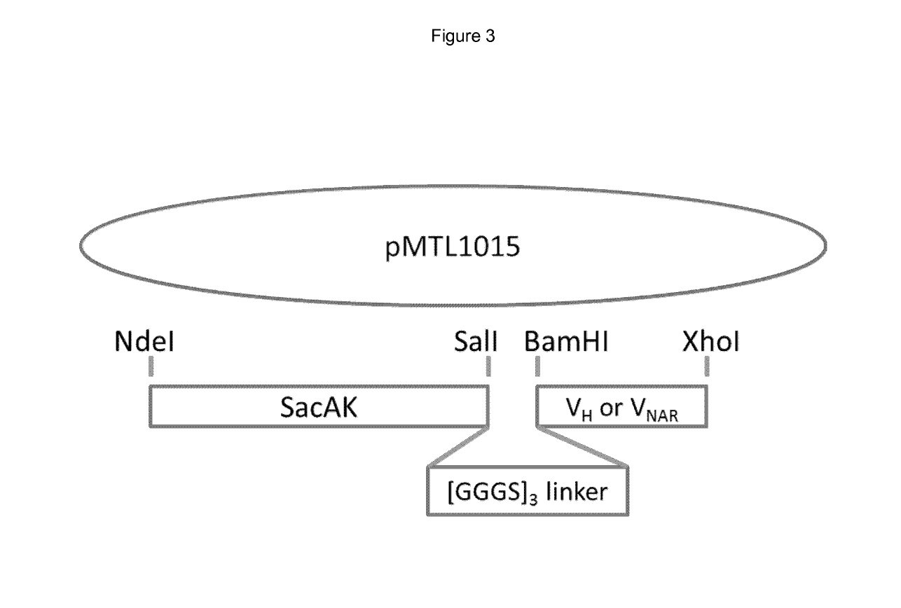

Conventional, commercially available expression systems can be used, typically a Gateway-adapted vector such as a pMTL1015 vector (Ungurs et al. 2010, as above). The Gateway-adapted vector illustrated in FIG. 3 has a cassette-based method of construction and can be used to rapidly generate alternative single domain antibody fusions. Other cassette-based vectors can also be used to construct the single domain antibody fusion proteins described herein.

The polynucleotide sequence can be expressed in the cytoplasm of a host cell using conventional techniques in the art.

Suitable host cells include prokaryotic and eukaryotic cells, preferably prokaryotic host cells such as E. coli. In one embodiment, recombination-defective host cells may be used, in order to minimise re-arrangement of the gene constructs during expression (eg. when two or more different fusion proteins are expressed in the same host cell). By way of example, a suitable recombination-defective host cell that may be used to express the fusion proteins of the invention is E. coli RV308.

Thus, in one aspect, the invention provides a method of producing a fusion protein of the invention as defined herein, comprising expressing a polynucleotide sequence encoding the fusion protein, or a plasmid comprising said polynucleotide sequence, in a host cell such as E. coli, and purifying the expressed fusion protein from the cytoplasm of the host cell.

In one embodiment of the invention, multiple different fusion proteins (which may be the same or different) are expressed in a single host cell. These fusion proteins can be expressed from a single plasmid using the same or different promoters. Alternatively, the fusion proteins can be expressed from two different compatible plasmids. The ratio of the fusion proteins expressed from the plasmid or plasmids may be varied by using high or low copy number plasmids, and/or high, medium or low expressing promoters (eg. as illustrated in Example 13 part (ii)). As discussed below, the expressed fusion proteins may multimerise to form a multimeric protein complex, which can be recovered from the host cell.

The invention also provides a method of preparing a single-domain antibody or single-domain antibody fragment, the method comprising:
(i) expressing the single-domain antibody or antibody fragment as a single-chain fusion protein with a thermostable kinase, in a host cell such as E. coli; and
(ii) purifying the fusion protein from the cytoplasm of the host cell.

All embodiments of the fusion proteins of the invention (as defined herein) apply equally to the corresponding polynucleotides (and plasmids, host cells etc.), and to the expression methods of the invention.

The expressed fusion protein may be extracted from the cytoplasm of the host cell (eg. E. coli) by any conventional technique known in the art. Typical extraction protocols may comprise lysing the host cells, and separating the soluble portion of the lysate by centrifugation.

The expressed, extracted fusion protein may be purified from the host cell lysate by any conventional technique known in the art.

In one embodiment, in view of the heat stability of the expression fusion protein, the purification step may involve high temperature treatment of the lysate (eg. at approx 80° C. for about 20 or 30 minutes). Following heat treatment, lysate may be clarified by removal of precipitated proteins. The purification step may comprise the step of applying the lysate to a chromatography column (eg. a Blue Sepharose column) and eluting the fusion protein using AMP and ATP.

Fusion proteins may be optionally further purified using an affinity column loaded with the ligand(s) for the single-domain antibody component(s). This step may be particularly useful to ensure that fusion proteins (or multimeric fusion protein complexes) comprising two or more different antibody components binding different ligands do indeed contain each of the different antibody components.

Functional recovery of correctly folded single-domain antibody or antibody fragment can be assessed in a conventional ELISA assay, using the thermostable kinase activity (eg. tAK activity) as the assay read-out.

As discussed above, thermostable kinases are known to form multimeric complexes.

As such, in one embodiment, the fusion proteins of the invention are recovered from the host cell as multimers (a multimeric fusion protein complex).

Formation of a multimeric fusion protein complex is due to multimerisation between the thermostable kinase components of multiple (ie. 2 or more, such as 2, 3, 4, 5, 6, 7 or 8) fusion proteins of the invention. By way of example, formation of a trimeric fusion protein complex is due to trimerisation between the thermostable kinase components (monomers) of 3 fusion proteins of the invention.

In one embodiment, the fusion proteins of the invention form multimers following recovery from the host cell (eg. during purification and refolding). The presence of high salt concentrations in the buffer used during the lysis phase may assist in the recovery of correctly folded proteins. Thus, in one embodiment, the lysis is carried out in a buffer solution containing 900 mM, 1000 mM, 1200 mM or 1500 mM NaCl, 500 mM, 700 mM 900 mM NH₄SO₄, or equivalents. The addition of organic solvents may also preferentially promote correct folding in the recovered proteins.

Thus, the invention provides a multimeric fusion protein complex comprising multiple (ie. 2 or more, such as 2, 3, 4, 5, 6, 7 or 8) fusion proteins of the invention.

The terms “multimer”/“multimeric” and the terms “polymer”/“polymeric” are used interchangeably herein and embrace, for example, dimers, trimers, pentamers, tetramers, hexamers, heptamers and octamers.

In one embodiment, a multimeric fusion protein complex of the invention is a dimeric fusion protein complex comprising two fusion proteins of the invention; or a trimeric fusion protein complex comprising three fusion proteins of the invention; or a tetrameric fusion protein complex comprising four fusion proteins of the invention; or a hexameric fusion protein complex comprising four fusion proteins of the invention; or an octomeric fusion protein complex comprising four fusion proteins of the invention.

A multimeric fusion protein complex of the invention thus comprises a thermostable kinase multimer (formed by multimerisation of thermostable kinase components of multiple fusion proteins of the invention), wherein each individual monomer of the thermostable kinase multimer is fused to a (or at least one) single-domain antibody or antibody fragment. In one embodiment of the present invention, each individual monomer of the thermostable kinase multimer may be fused to two or more single-domain antibodies or antibody fragments, which may be the same, or may be different (as discussed above).

By way of example, the multimeric fusion protein complex may comprise a thermostable kinase dimer, wherein each of the two thermostable kinase monomers of the dimer is fused to a single-domain antibody or antibody fragment. The fusion protein may comprise a thermostable kinase trimer, wherein each of the three thermostable kinase monomers is fused to a single-domain antibody or antibody fragment. The fusion protein may comprise a thermostable kinase tetramer, wherein each of the four thermostable kinase monomers is fused to a single-domain antibody or antibody fragment. The fusion protein may comprise a thermostable kinase hexamer, wherein each of the six thermostable kinase monomers is fused to a single-domain antibody or antibody fragment. The fusion protein may comprise a thermostable kinase octomer, wherein each of the eight thermostable kinase monomers is fused to a single-domain antibody or antibody fragment.

Alternatively, the multimeric fusion protein complex may comprise a thermostable kinase dimer, wherein each of the two thermostable kinase monomers of the dimer is fused to two or more single-domain antibodies or antibody fragments, which may be the same, or may be different. The fusion protein may comprise a thermostable kinase trimer, wherein each of the three thermostable kinase monomers is fused two or more single-domain antibodies or antibody fragments, which may be the same, or may be different. The fusion protein may comprise a thermostable kinase tetramer, wherein each of the four thermostable kinase monomers is fused to two or more single-domain antibodies or antibody fragments, which may be the same, or may be different. The fusion protein may comprise a thermostable kinase hexamer, wherein each of the six thermostable kinase monomers is fused to two or more single-domain antibodies or antibody fragments, which may be the same, or may be different. The fusion protein may comprise a thermostable kinase octomer, wherein each of the eight thermostable kinase monomers is fused to two or more single-domain antibodies or antibody fragments, which may be the same, or may be different.

The fusion proteins of the invention that make up the multimeric fusion protein complex of the invention may be the same or different (ie. the multimeric complexes may be homo-multimeric or hetero-multimeric). For example, the multimeric fusion protein complex of the invention may be homo-dimeric, homo-trimeric, homo-tetrameric, homo-hexameric or homo-octomeric. Alternatively, the multimeric fusion protein complex of the invention may be hetero-dimeric, hetero-trimeric, hetero-tetrameric, hetero-hexameric or hetero-octomeric.

In one embodiment, the single-domain antibodies or antibody fragments within the multimeric fusion protein complex are all the same. Thus, the same single-domain antibodies or antibody fragments are fused to each of the individual thermostable kinase monomers.

In an alternative embodiment, the single-domain antibodies or antibody fragments that are fused to the individual monomers of the thermostable kinase within the multimeric complex are not all the same. In accordance with this embodiment, the multimeric fusion protein complex comprises a mixture of two or more different single-domain antibodies/antibody fragments (eg. 2, 3, 4, 5, 6, 7 or 8 different single-domain antibodies/antibody fragments), each fused to individual thermostable kinase monomers. By way of example, the different antibodies/antibody fragments may be located within the same fusion protein, or in different fusion proteins.

The different single-domain antibodies/antibody fragments may differ from each other in terms of their binding activity. In this regard, the different single-domain antibodies or antibody fragments fused to different thermostable kinase monomers may bind to different analytes, or may bind to the different epitopes of the same analyte, or may bind to the same analyte/epitope but with different affinities. In one embodiment, the different analytes bound by the single-domain antibodies or antibody fragments may be (or be derived from/components of) different biological infectious or non-infectious agents (eg. different bacterial or viral antigens). In one embodiment, the different analytes bound by the single-domain antibodies or antibody fragments may be a combination of (i) analytes from one or more different biological infectious/non-infectious agents and (ii) analytes having a detector molecule function.

In one embodiment, all the fusion proteins within the multimeric fusion protein complex are the same (ie. a homo-multimeric fusion protein complex is formed).

In an alternative embodiment, the fusion proteins within the multimeric fusion protein complex are not all the same (ie. a hetero-multimeric fusion protein complex is formed). In accordance with this embodiment, the multimeric fusion protein complex comprises a mixture of two or more different fusion proteins (eg. 2, 3, 4, 5, 6, 7 or 8 different fusion proteins). The different fusion proteins may be expressed either from one plasmid using the same promoter or from one plasmid using different promoters. Alternatively, the different fusion proteins may be expressed from two different (compatible) plasmids.

The ratio of different fusion proteins present in the hetero-multimeric fusion protein complex of the invention will depend on the relative abundance of fusion proteins expressed, which may be varied by using high or low copy number plasmids, and/or high, medium or low expressing promoters (eg. as discussed in Example 13 part (ii)).

All embodiments of the fusion proteins of the invention (as defined herein) apply equally to multimeric complexes thereof, and to the extraction and purification methods of the invention.

In one embodiment, the fusion protein of the invention may be detected by detecting the kinase activity of the thermostable kinase.

Generally, the thermostable kinase is detected using a substrate comprising ADP. The kinase activity of the thermostable kinase converts the ADP to ATP, which is itself used to generate light (eg. by reaction with a bioluminescent reagent).

The term “bioluminescent reagent” refers to any substance or mixture of substances able to react with ATP to generate light, such as a mixture of luciferin and luciferase. Standard luciferin-luciferase assay methods can detect as little as 10⁻¹⁵ moles of ATP. By coupling an enzymatic amplification to the bioluminescent detection methods it is possible to detect as few as 10⁻²⁰ moles of kinase. The light emitted by the reaction of ATP with the bioluminescent reagent can be measured using conventional techniques known in the art, such as using a standard luminometer (eg. a Berthold Orion 96-well microplate luminometer, or a hand-held luminometer).

By way of example, the thermostable kinase can be reacted with ADP at a temperature of between 30° C. and 70° C., and the formation of ATP can be detected and measured by bioluminescent detection using luciferin/luciferase and a suitable luminometer at 20-30° C. for 10 minutes to 1 hour.

The data generated by luminometers is expressed in “Relative Light Units” (RLUs). RLUs are a relative, not absolute, measurement. To address this issue, manufacturers have generated data for RLU “factors”, which allow the data generated by a given luminometer to be normalised to a calibrated standard. Thus, comparisons can be made between different instruments. The figures given in the present specification relate to measurements taken using a Berthold Orion 96-well microplate luminometer with injector system using a “flash” method of light measurement for 2 seconds immediately after the addition of the luciferase/luciferin reagents (technical specification photomultiplier measuring light emitted at a wavelength of 300-650 nm). The RLU factor for the Berthold Orion 96-well microplate luminometer is 1. Accordingly, the RLU values given in the specification can be regarded as standardised/normalised RLU values.

In terms of absolute values, an RLU value can be related to the concentration of ATP required to give said value with the reagents as described in the method. As an approximate conversion, and given the linear relationship between RLU values and ATP concentration, the following values can be used:

| RLU | Approximate concentration of ATP/μM |
|---|---|
| 12,000,000 | 1000 |
| 1,200,000 | 100 |
| 120,000 | 10 |
| 12,000 | 1 |
| 1,200 | 0.1 |
| 120 | 0.01 |

In one embodiment, the thermostable kinase component of the fusion protein of the invention has an activity of at least 500,000 RLU per mg kinase, or at least 1,000,000 RLU per mg kinase, or at least 3,000,000 per mg kinase, or at least 5,000,000 RLU per mg kinase, or at least 8,000,000 RLU per mg kinase, or at least 10,000,000 Relative Light Units (RLU) per mg kinase, when measured in the presence of luciferin/luciferase by a luminometer.

Those familiar with the art will recognise other methods for the detection of ATP as the product of a thermostable kinase reaction. These might include detection using absorbance measurement via HPLC, or chemiluminescent, fluorescent or colorimetric measurement using suitable reactive compounds.

In one embodiment of the present invention, the sensitivity of the ATP detection reaction may be further increased by providing one or more additional single-domain antibodies or antibody fragments that bind and recruit one or more additional thermostable kinases to the antibody-analyte complexes (ie. in addition to the antibody and thermostable kinase component(s) present in the fusion protein). Examples of this embodiment of the invention are presented in Examples 12 and 13. The additional single domain antibody/antibody fragment may be present in the fusion protein targeting the analyte of interest (ie. the analyte being detected). Alternatively, the additional single domain antibody/antibody fragment may be present in a different fusion protein from the fusion protein targeting the analyte(s) of interest. The fusion protein comprising the single domain antibody/antibody fragment that binds and recruits an additional thermostable kinase can be present in the form of a single fusion protein, or as part of a homo-multimeric or hetero-multimeric fusion protein complex.

Optionally, the fusion protein that targets the analyte(s) of interest and the fusion protein that binds and recruits the additional thermostable kinase(s) are present within the same hetero-multimeric fusion protein complex.

The additional thermostable kinase recruited to the antibody-analyte complexes (by the additional single domain antibody/antibody fragment) may, for example, be any of the thermostable kinases described herein.

In one embodiment of the present invention, one or more alternative or additional “detector molecules” (as defined herein) may be used to detect the antibody-analyte complexes formed by the fusion protein complexes of the present invention (in addition to or instead of an ATP detection step) eg. as discussed in Example 14 part (ii).

By way of example, the detection step may employ one or more additional single domain antibodies or antibody fragments that bind and recruit the detector molecule(s) to the antibody-analyte complexes. The additional single domain antibody/antibody fragment targeting the detector molecule may be present in the fusion protein targeting the analyte of interest (ie. the analyte being detected). Alternatively, the additional single domain antibody/antibody fragment may be present in a different fusion protein from the fusion protein targeting the analyte(s) of interest. The fusion protein that binds and recruits the additional detector molecule can be present in the form of a single fusion protein or as part of a homo-multimeric or hetero-multimeric fusion protein complex.

Optionally, the fusion protein that targets the analyte(s) of interest and the fusion protein that binds and recruits the additional detector molecule(s) are present within the same hetero-multimeric fusion protein complex.

Examples of suitable detector molecules are described herein (eg. a fluorophore). The detector molecules described herein can be detected using any conventional means known in the art.

WO 00/46357 describes detection of analytes using an antibody conjugated to a ‘reporter’ thermostable kinase.

The fusion protein of the present invention may be used as a reporter molecule in a detection assay for identifying and/or quantifying an analyte to which the single-chain antibody or antibody fragment binds.

Thus, in one aspect, the invention provides a reporter molecule for detecting an analyte on or within a sample, the reporter molecule comprising a fusion protein of the invention as defined herein.

The reporter molecule of the invention may comprise multiple (eg. 2, 3, 4, 5, 6, 7 or 8), different fusion proteins of the invention. In this regard, the fusion proteins may comprise different thermostable kinases and/or different single-domain antibodies/antibody fragments. By way of example, the different single-domain antibodies/antibody fragments may bind different analytes, or may bind to the same or different epitopes on the same analyte.

In one embodiment, the receptor molecule of the invention comprises a multimeric fusion protein complex of the invention, as defined above.

The invention also provides an in vitro method of detecting an analyte on or within a sample, comprising:
(i) contacting the sample with a fusion protein or reporter molecule of the invention as defined herein, wherein the single-domain antibody or antibody fragment binds the analyte to form an antibody-analyte complex; and
(ii) detecting the antibody-analyte complex.

Detection of the antibody-analyte complex indicates that the analyte is present on or within the sample. If antibody-analyte complex is not detected, this indicates that the analyte is not present on or within the sample.

In accordance with this aspect of the invention, the fusion protein binds the analyte via the single-domain antibody or antibody fragment, to form an antibody-analyte complex. If antibody-analyte complexes are detected, this indicates that the fusion protein of the invention bound to analyte—ie. there was analyte present in the sample. If antibody-analyte complexes are not detected, this indicates that the fusion protein did not bind to analyte—ie. there was no analyte present in the sample.

One advantage of reporter molecules comprising fusion proteins or multimeric fusion protein complexes described herein that comprise multiple (eg. at least two) different antibody components binding different analytes is the ability to use a single reagent to simultaneously detect multiple different analytes.

The invention thus provides an in vitro method of detecting multiple (eg. at least 2, 3, 4, 5, 6, 7 or 8) different analytes on or within a sample, comprising:
(i) contacting the sample with a fusion protein or reporter molecule comprising multiple (eg. at least two) antibody components, wherein said antibody components bind to said multiple different analytes to form antibody-analyte complexes; and
(ii) detecting the antibody-analyte complexes.

Detection of the antibody-analyte complexes indicates that the analytes are present on or within the sample.

Viruses (such as the influenza virus or norovirus) have the ability to rapidly mutate and undergo a shift in antigen diversity. Detection of virus infection is typically performed (in the art) using a single monoclonal antibody that targets a single virus antigen. Mutation of the antigen can prevent binding of the monoclonal antibody leading to a false negative signal. Polyclonal antibodies can be used to address this problem; however, they are slow to produce, and variable in quality.

In one embodiment, the present invention overcomes this problem by providing a single reagent comprising multiple different antibody components that recognise multiple different antigens (eg. multiple different viral antigens). It is unlikely that all of the different target antigens will undergo mutation to evade detection by the antibody/antibody fragment, and so the fusion proteins/fusion protein complexes of the present invention advantageously reduce the risk of false negative results when determining (for example) the presence of a virus infection. An example of this aspect of the invention is described in Example 13 part (i).

The fusion proteins/fusion protein complexes and reporter molecules described herein are also useful for detecting multiple different virus genotypes using a single reagent (where the different single domain antibody components of the fusion proteins/fusion protein complexes bind to different virus genotypes). By way of example, a fusion protein/fusion protein complex or reporter molecule of the invention as described herein are useful for simultaneously detecting the presence of the norovirus genotypes GI and GII (eg. as discussed in Example 13 part (i)).

The invention thus provides an in vitro method of detecting multiple (eg. at least 2, 3, 4, 5, 6, 7 or 8) different viral genotypes (eg. norovirus genotypes GI and GII) on or within a sample, comprising:
(i) contacting the sample with a fusion protein or reporter molecule of the invention comprising multiple (eg. at least 2, 3, 4, 5, 6, 7 or 8) antibody components, wherein said antibody components bind to viral antigens from said multiple different genotypes to form antibody-antigen complexes; and
(ii) detecting the antibody-antigen complexes.

Detection of the antibody-antigen complexes indicates that the viral genotypes are present on or within the sample.

The ability to simultaneously detect multiple analytes using a single reagent is also an advantage when detecting the presence of bacterial infections—for example, for detecting the presence of multiple different bacterial infections, or for detecting the presence of a single bacterial infection associated with multiple different toxins that can mediate disease either individually or in combination. For example, Clostridium difficile can mediate disease through the expression of toxin B alone or in combination with toxin A. Hence, in one embodiment, a fusion protein/fusion protein complex or reporter molecule of the invention as defined herein is useful to screen clinical isolates to detect the presence of Clostridium difficile expressing only toxin A or toxin B, or a combination of both toxin A and toxin B (eg. as discussed in Example 13 part (i)).

The invention thus provides an in vitro method of detecting multiple (eg. at least 2, 3, 4, 5, 6, 7 or 8) different bacterial proteins (eg. C. difficile proteins such as toxin A and/or toxin B) on or within a sample, comprising:
(i) contacting the sample with a fusion protein or reporter molecule comprising multiple (eg. at least 2, 3, 4, 5, 6, 7 or 8) antibody components, wherein said antibody components bind to said multiple different bacterial proteins to form antibody-protein complexes; and
(ii) detecting the antibody-protein complexes.

Detection of the antibody-protein complexes indicates that the bacterial proteins are present on or within the sample.

The term “sample” encompasses any item, instrument, surface, fluid or material. Examples include, but are not limited to, clinical samples (such as blood (eg. clarified whole blood), serum, sputum, oral samples such as saliva, pus, vaginal samples, stool samples, vomit); environmental samples (such water, soil, air samples); surgical and medical equipment and instruments such as endoscopes, microtitre plates, dipsticks, lateral flow devices, hospital gowns, bedclothes; bulk liquids; culled animal material; pharmaceuticals; workbenches, walls and floors.

Reference to “contacting the sample” with a fusion protein embraces contacting a portion or an extract of the sample suspected of comprising the analyte.

Prior to carrying out the method of the invention, it is not necessarily known whether analyte is present on or within the sample. It is possible that the sample may not contain any analyte at all. In general, however, the analyte is known to be present on or within the sample. In one embodiment, the aim of the assay is to identify whether or not analyte is present on or within the sample. In one embodiment, the aim of the assay is to quantify analyte known to be present on or within the sample.

In one embodiment, the method of the invention comprises the step of treating the sample in order to (substantially) remove any endogenous kinase present on or within the sample. This treatment step may be conducted prior to, after, or simultaneously with the step of contacting the sample with the fusion protein or reporter molecule of the invention, so long as it is conducted prior to the step of detecting the antibody-analyte complex by detecting the kinase activity of the thermostable kinase. In one embodiment, the removal of endogenous kinase is performed prior to contacting the sample with the fusion protein or reporter molecule of the invention. This treatment step ensures that the signal obtained from the assay is not contaminated or otherwise adversely affected by any endogenous kinase present in the sample being tested (ie. reduces the risk of “false positive” readings).

Reference to “removing kinase” from the sample embraces removing kinase activity, such as by denaturing or otherwise inactivating any endogenous kinase in situ. By way of example, removal of endogenous mesophilic kinase can conveniently be achieved by heating the sample to a temperature at which any endogenous mesophilic kinase (if present) is denatured or rendered incapable of catalysing the formation of ATP, but at which the thermostable kinase present in the fusion protein of the invention is not denatured and retains its kinase activity. A suitable treatment might be to heat the sample to 60 to 90° C. for at least 10 minutes (such as 70° C. for at least 30 minutes, or at 80° C. for at least 10 minutes). Alternatively, other treatments might be appropriate to destroy the activity of endogenous kinase, whilst retaining activity of the thermostable kinase of the invention, such as the use of ultrasound, or extremes of pH or salt concentration.

In one embodiment, after the step of contacting the sample with the fusion protein or reporter molecule of the invention, and before the step of detecting the antibody-analyte complex, the method of the invention comprises removing any fusion protein that is not bound to analyte. This step ensures that the signal obtained from the assay is not contaminated or otherwise adversely affected by kinase added to the sample that does not bind the analyte of interest (ie. reduces the risk of “false positive” readings).

In one embodiment, antibody-analyte complexes may be detected by detecting the kinase activity of the thermostable kinase component of the fusion protein.

The kinase activity of the thermostable kinase can be measured by any conventional means known in the art. In one embodiment, the thermostable kinase component of the fusion protein provides a highly specific output in the form of ATP generation and hence light generation, which allows for highly sensitive and specific detection of the binding event between the antibody/antibody fragment and the analyte. The detection limits are at or below the dissociation constant (Kd) of the antibody, providing a highly sensitive assay.

In one embodiment, the kinase activity of the bound thermostable kinase in the antibody-analyte complex is detected by adding a substrate (eg. ADP), and detecting the formation of ATP. The ATP may be detected and measured directly, but is more usually detected indirectly, eg. by reaction of the ATP with a bioluminescent reagent such as luciferin/luciferase, to generate light. The light output from the assay (ie. the light emitted by the reaction of ATP with the bioluminescent reagent) can be measured using conventional techniques known in the art, such as using a luminometer.

Detection of ATP indicates that the assay contained the fusion protein of the invention bound to analyte—ie. analyte was present in the sample. If no ATP is detected, this indicates that the fusion protein did not bind to analyte—ie. analyte was not present in the sample.

In one embodiment, prior to addition of ADP and detection of kinase activity, the method comprises the step of removing any endogenous ATP present on or within the sample. This step further decreases the background noise in the assay. The removal of endogenous ATP may be achieved by any conventional method known in the art—for example, by adding an ATPase such as a thermolabile ATPase that can itself be destroyed by use of elevated temperature (to avoid the ATPase adversely influencing the signal obtained from the thermostable kinase).

In one embodiment, the method comprises the following steps:
1. A sample containing or suspected of containing an analyte of interest is contacted with a fusion protein or reporter molecule of the invention comprising a single-domain antibody or antibody fragment that binds the analyte of interest;
2. The fusion protein binds to analyte in the sample (if present) via the single-domain antibody or antibody fragment, to form antibody-analyte complexes;
3. The assay is treated (eg. by washing) to remove unbound components of the sample and unbound fusion protein, but so as to retain any antibody-analyte complexes;
4. Optionally, a thermolabile ATPase is added to the solid phase to remove any endogenous ATP;
5. The assay is treated (eg. by heating) to destroy the thermolabile ATPase (if added) and to denature any endogenous kinase that may be present, but so as not to denature the thermostable kinase in the fusion protein;
6. ADP is added to the assay, as a substrate for the thermostable kinase; and the assay is tested for presence and/or amount of ATP (eg. by adding a bioluminescent compound and detecting/measuring the light output).

In one embodiment, the ATP detection signal generated by the antibody-analyte complexes is further amplified using one or more additional single domain antibodies or antibody fragments that bind and recruit one or more additional thermostable kinase(s) to the antibody-analyte complexes.

In one embodiment, the additional single domain antibody/antibody fragment may be present in the fusion protein targeting the analyte of interest (ie. the analyte being detected). Alternatively, the additional single domain antibody/antibody fragment may be present in a different fusion protein from the fusion protein targeting the analyte(s) of interest. The fusion protein comprising the single domain antibody/antibody fragment that binds and recruits an additional thermostable kinase can be present in the form of a single fusion protein, or as part of a homo-multimeric and hetero-multimeric fusion protein complex.

Optionally, the fusion protein that targets the analyte(s) of interest and the fusion protein that binds and recruits the additional thermostable kinase(s) are present within the same hetero-multimeric fusion protein complex.

In an alternative embodiment of the present invention, one or more alternative or additional “detector molecules” may be used to detect the antibody-analyte complexes formed by the fusion protein complexes of the present invention (in addition to or instead of an ATP detection step) eg. as discussed in Example 14 part (ii).

By way of example, the detection step may employ one or more additional single domain antibodies or antibody fragments that bind and recruit the detector molecule(s) to the antibody-analyte complexes. The additional single domain antibody/antibody fragment targeting the detector molecule may be present in the fusion protein targeting the analyte of interest (ie. the analyte being detected). Alternatively, the additional single domain antibody/antibody fragment may be present in a different fusion protein from the fusion protein targeting the analyte(s) of interest. The fusion protein comprising the single domain antibody/antibody fragment that binds and recruits an additional detector molecule can be present in the form of a single fusion protein or as part of a homo-multimeric or hetero-multimeric fusion protein complex.

Optionally, the fusion protein that targets the analyte(s) of interest and the fusion protein that binds and recruits the additional detector molecule(s) are present within the same hetero-multimeric fusion protein complex.

Examples of suitable detector molecules are described herein (eg. a fluorophore). The detector molecules described herein can be detected using any conventional means known in the art.

In one embodiment, detection of the detector molecule is performed in addition to detecting the kinase activity of the thermostable kinase component of the fusion protein (and/or the kinase activity of any additional thermostable kinase recruited to the antibody-analyte complex). Alternatively, detection of the detector molecule may be performed instead of detecting the kinase activity of the thermostable kinase component of the fusion protein (and/or the kinase activity of any additional thermostable kinase recruited to the antibody-analyte complex).

In one embodiment of the present invention, the additional reporter or tracer molecules recruited to the antibody-analyte complexes may be used to detect the presence of the analyte(s) of interest in a whole body system or in isolated tissue samples by histology.

The term “analyte” encompasses both infectious and non-infectious agents derived from a biological source. Examples of analytes include bacteria, viruses, fungi, prions, toxins, allergens, spores, and fragments and derivatives/components of any of the foregoing. In one embodiment, an analyte may be referred to as a ‘contaminant’ or a ‘contaminating biological agent’.

The present invention is applicable to detection of any analyte against which a single-domain antibody can be raised. In other words, any analyte can be detected using the present invention, so long as it can be bound by the fusion protein of the invention via the single-domain antibody or antibody fragment.

In one embodiment, the analyte comprises or consists of one or more proteins, nucleic acids, carbohydrates and/or lipids. In one embodiment, the analyte comprises or consists of a virus, or a cell such as a bacterium or yeast.

In one embodiment, the analyte is derived from a pathogen, such as a pathogenic bacterium or virus.

In one embodiment, the analyte comprises or consists of a protein, such as a protein selected from the group consisting of an animal (eg. mammal such as human) protein, a bacterial protein, a viral protein, a plant protein or a fungal protein. In one embodiment, the analyte comprises or consists of a blood protein or a protein derived from neurological tissue.

In one embodiment, the analyte comprises or consists of a bacterial protein from a bacterial species that may be a pathogen of humans, animals or plants and may be found in clinical samples, environmental samples, or process samples. The bacterial protein may, for example, be selected from the group consisting of a bacterial fimbrial protein (eg. CgsA from E. coli and AgfA from Salmonella), a bacterial toxin protein (eg. toxins from Bacillus species, such as Bacillus anthracis, Corynebacterium diphtheriae, Clostridial species, such as Clostridium botulium (eg. C. botulinum neurotoxin (BoNT)) and Clostridium difficile (eg. C. difficile toxin A and toxin B), Staphylococcus species, such as Staphylococcus aureus (eg. S. aureus enterotoxin B)), a bacterial enzyme, such as a bacterial metalloprotease (eg. New Delhi metalloprotease produced by E. coli and Klebsiella); a bacterial cell surface protein (eg. peptidoglycan, lipoproteins); and a bacterial spore protein (eg. from Gram positive bacteria).

In one another embodiment, the analyte comprises or consists of a viral protein from a virus that may be a pathogen of humans, animals, bacteria or plants and may be found in clinical samples, environmental samples, or process samples. The viral protein may be a nucleoprotein, a viral envelope protein, a viral capsid protein, or a viral core protein. Examples of viral proteins are from a bacteriophage virus (eg. the MS2 and PP7 proteins), norwalk virus (eg. capsid protein), rotavirus (eg. VP2, VP6 and VP7 proteins), coronavirus (eg. SARS S, E and M proteins), bluetongue virus (eg. VP2 protein), human papillomavirus (eg. viral major structural protein, L1), hepatitis B (eg. small envelope protein HBsAg), Hepatitis C virus (eg. core, E1 and E2 proteins), influenza virus (eg. neuraminidase and haemagglutinin and matrix proteins), poliovirus (eg. capsid VP0, 1 and 3 proteins), HIV (eg. Pr55gag, envelope proteins), dengue B virus (eg. envelope (e) and pre-membrane/membrane (prM/M), ebola virus and norovirus.

In another embodiment, the analyte comprises or consists of a fungal protein selected from the group consisting of cell wall proteins (eg. SC3 from Schizophyllum commune, RodA/B from Aspergillus fumigates, and equivalent proteins from yeast), fungal spore proteins, hyphal proteins, mycotoxins, and fungal prions (eg. Sup35, Het S, URE 2, Rnq1, New 1).

In one embodiment, the analyte comprises or consists of a blood protein selected from the group consisting of blood clotting proteins (eg. fibrinogen, fibrin peptides, fibrin, transglutaminase substrates, thrombin), serum proteins (e.g. albumin and globulin), platelet proteins, blood cell glycoproteins, factor VIII, and haemoglobin.

In yet another embodiment, the analyte comprises or consists of a self-aggregating protein selected from the group consisting of prions (eg. PrP^{sc} and PrP^{c}, Sup35, Het S, Ure 2, Rnq1, New 1), prion mimetic proteins, amyloid fibrils, (eg. amyloid beta), and misfolded proteins (eg. alpha-synuclein).

In one embodiment, the analyte comprises or consists of a small molecule that is a substance of abuse. Examples of such molecules include nicotine and cocaine.

In another embodiment, the analyte comprises or consists of a nucleic acid molecule, such as a DNA molecule or an RNA molecule. By way of example, the nucleic acid analyte may comprise or consist of single-stranded DNA (ssDNA), single-stranded RNA (ssRNA), double-stranded DNA (dsDNA) or double-stranded RNA (dsRNA). In one embodiment, the nucleic acid molecule comprises or consists of a nucleic acid sequence that encodes any of the protein analytes defined above.

In another embodiment, the analyte comprises or consists of a carbohydrate, such as a carbohydrate selected from the group consisting of exopolysaccharide, lipopolysaccharide (EPS/LPS, sometimes known as endotoxin) (eg. from Legionella species, E. coli, Staphylococcus species, Streptococcus species, Pseudomonas species, Acinetobactor species, Campylobactor species, and Bacillus species), peptidoglycan, cell wall components of plants, fungi and yeast (eg. chitin, lignin, glucan), mucin preparations, glycolipids (eg. brain derived glycolipids), glycoproteins (eg. cell surface glycoproteins, Eap1p), spore extracts (eg. from Bacillus spp, Clostridial spp and other spore-formers), polysaccharides from yeast capsules, and invertebrate secretions (eg. from molluscan gels).

In another embodiment, the analyte comprises or consists of a lipid, such as a lipid selected from the group consisting of glycolipids (eg. brain-derived glycolipids), gangliosides (eg. neuronal cell gangliosides such as GT_{1b}, GT₁ₐ and gangliosides of more general cell origin such as GM₁), and plant oils and lipids.

In another embodiment, the analyte may be derived from or may comprise/consist of one or more components of blood, serum, albumin, mucus, egg, neurological tissue, food, culled animal material, or one or more components present in soil, water or air samples. In yet another embodiment of the invention, the biological matrix comprises one or more components selected from the group consisting of fibrinogen, thrombin, factor VIII, CaCl₂, and, optionally, albumin and/or haemoglobin.

In another embodiment, the term “analyte” also encompasses molecules having a detector function, which are provided as part of the detection reaction, as discussed above. In the present invention, analytes having a detector function (“detector molecules”) may be provided to increase the sensitivity of the detection reaction for detection of biological analyte(s) of interest (eg. as discussed above).

In one embodiment, a detector molecule may comprise or consist of a thermostable kinase (such as an adenylate kinase, acetate kinase, UMP kinase, pyruvate kinase or butyrate kinase or any thermostable kinase described herein).

In another embodiment, the detector molecule may comprise or consist of a fluorophore, hapten (eg. biotin or digoxigenin), phosphatase, peroxidase, acridine ester, radioisotope (eg. 32P, 35S) or any other conventional reporter molecule known in the art. In another embodiment, the detector molecule may comprise or consist of a tracer, such as an isotopic tracer (eg. ²H, ¹³C, and ¹⁵N), or any other conventional tracer molecule known in the art.

The fusion proteins may be used in a variety of assay formats familiar to those with knowledge of the art. Accordingly, in one embodiment, the invention provides a fusion protein or a reporter molecule of the invention, wherein the fusion protein is attached to a solid support. Suitable examples of assay formats include an enzyme-linked immunoassay (ELISA) or variant thereof, a magnetic bead-based solid support assay, a Western Blot, an autoanalyser, a nucleic acid amplification system, a lateral flow or dipstick assay or other rapid assay format.

In a further aspect of the invention, there is provided a detection kit comprising: (i) one or more fusion proteins, fusion protein complexes or reporter molecules of the invention as defined herein, and (ii) a substrate for the thermostable kinase.

In one embodiment, the kit comprises a multimeric fusion protein complex comprising multiple fusion proteins of the invention (as defined herein).

In one embodiment, the substrate for the thermostable kinase is ADP.

In one embodiment, to measure the thermostable kinase amount or activity, the kit may also include means for detecting ATP, such as a bioluminescent reagent (e.g. luciferin/luciferase) and optionally a luminometer.

From previous testing with known analytes, data can be prepared correlating the amount of kinase activity with the amount or activity of the analyte. The kit may therefore further comprise a look-up table correlating the observed kinase activity with the amount or activity of a list of specified analytes.

In one embodiment, the kit may further comprise one or more detector molecules (as defined herein) that can be used to detect antibody-analyte complexes in addition to (or instead of) detecting the thermostable kinase activity of the fusion protein. In one embodiment, the additional reporter molecule may comprise or consist of one or more additional thermostable kinases, fluorophores, biotin, digoxigenin, phosphatase, peroxidase, and other conventional reporter or tracer molecules known in the art. The kit may also comprise conventional means for detecting the detector molecule(s).

All references cited in this application are hereby incorporated by reference in their entirety.

The invention is now described in specific embodiments in the following examples and with reference to the accompanying drawings.

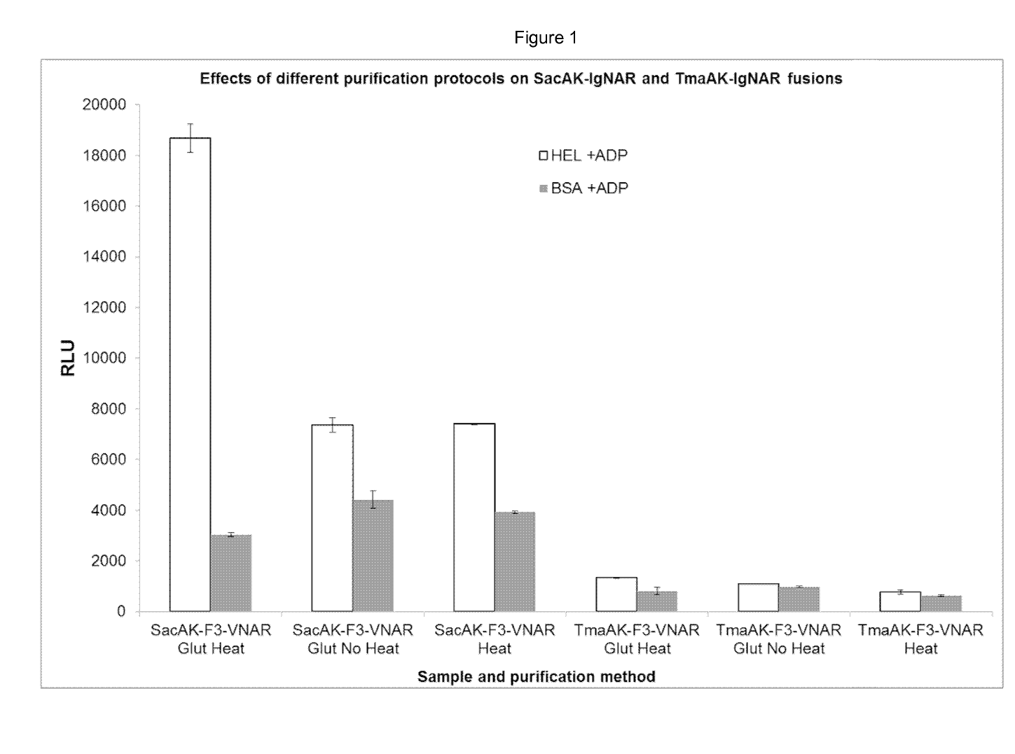

FIG. 1 illustrates functional recovery of correctly folded single-domain antibody (V_{NAR}) in the presence of the Sac tAK domain and glutathione. The fusion proteins were assessed for their ability to bind to hen egg lysozyme immobilized onto a microtitre plate in an ELISA format, using the tAK activity as the assay read-out. In each case background binding was measured using BSA as a non-relevant antigen control. In the case of Tma tAK fusions, no significant increase in binding was observed above the background binding to BSA with either glutathione extraction, heat treatment or a combination of the two. In contrast, for the Sac tAK fusions, heat or extraction in the presence of glutathione resulted in an increase in productive binding above the BSA control, whilst the combination of the two factors gave approximately 10-fold increase in binding.

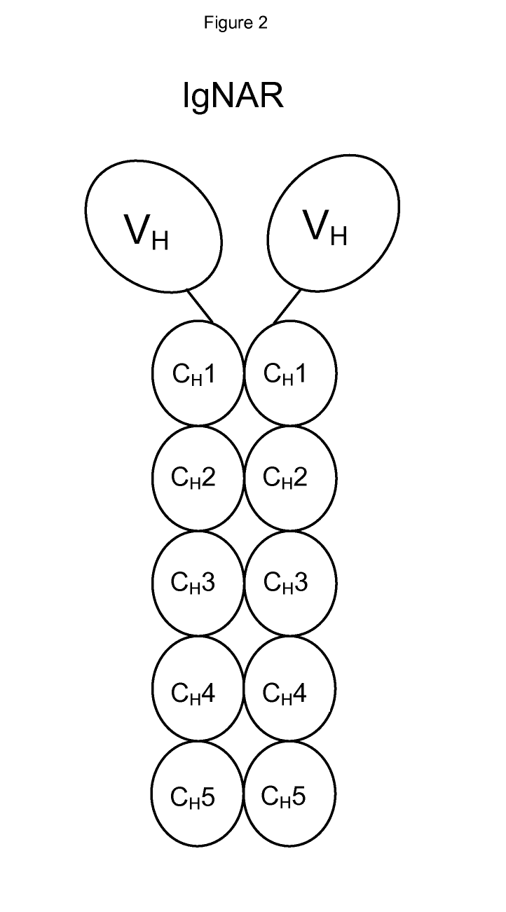

FIG. 2 provides a diagrammatic representation of shark Ig new antigen receptors (V_{NAR}).

FIG. 3 illustrates construction of a SacAK-F3-V_{NAR} fusion or SacAK-F3-V_{H} fusion. SacAK was cloned into a Gateway-adapted pMTL1015 vector (Ungurs et al. 2010), upstream of the recombination site. The V_{NAR} or V_{H} was recombined from an entry vector to generate the first generation construct. Where required, additional linker regions (eg. (GGGGS)₃) were cloned to replace the intergene region using engineered restriction enzyme sites as shown.

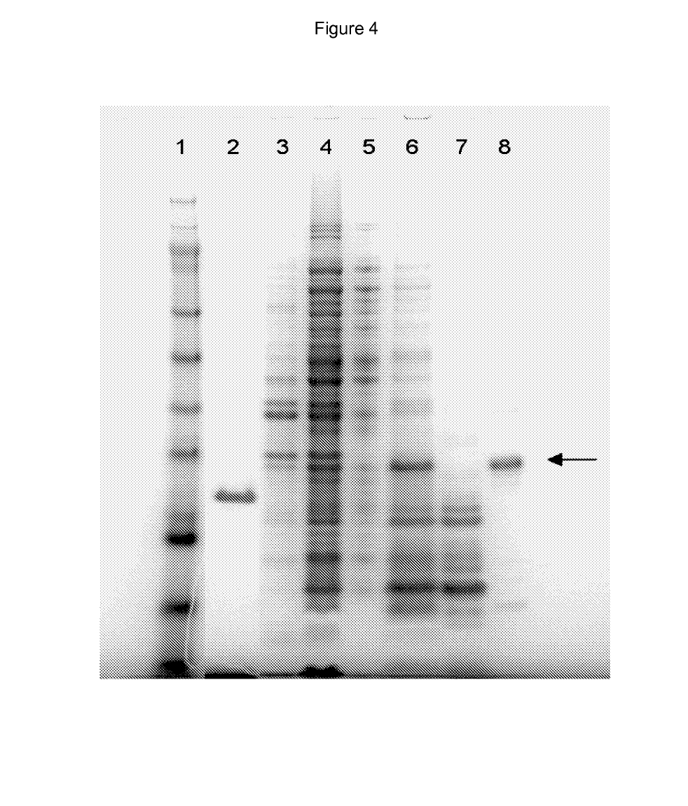

FIG. 4 illustrates SDS page analysis of purification of SacAK-F3-V_{NAR} fusion. Equal amounts of protein from each stage of the purification was loaded onto a 4-12% Bis Tris gel and electrophoresed in MES buffer under standard conditions. Purified SacAK-F3-VNAR is indicated with an arrow and lanes loaded as follows: 1: See Blue plus 2 ladder, 2: SacAK control (2.5 μg), 3: Insoluble homogenate, 4: Soluble homogenate, 5: Insoluble thermoprecipitate, 6: Soluble thermoprecipitate/column load, 7: Blue Sepharose column wash, 8: Blue Sepharose column eluate

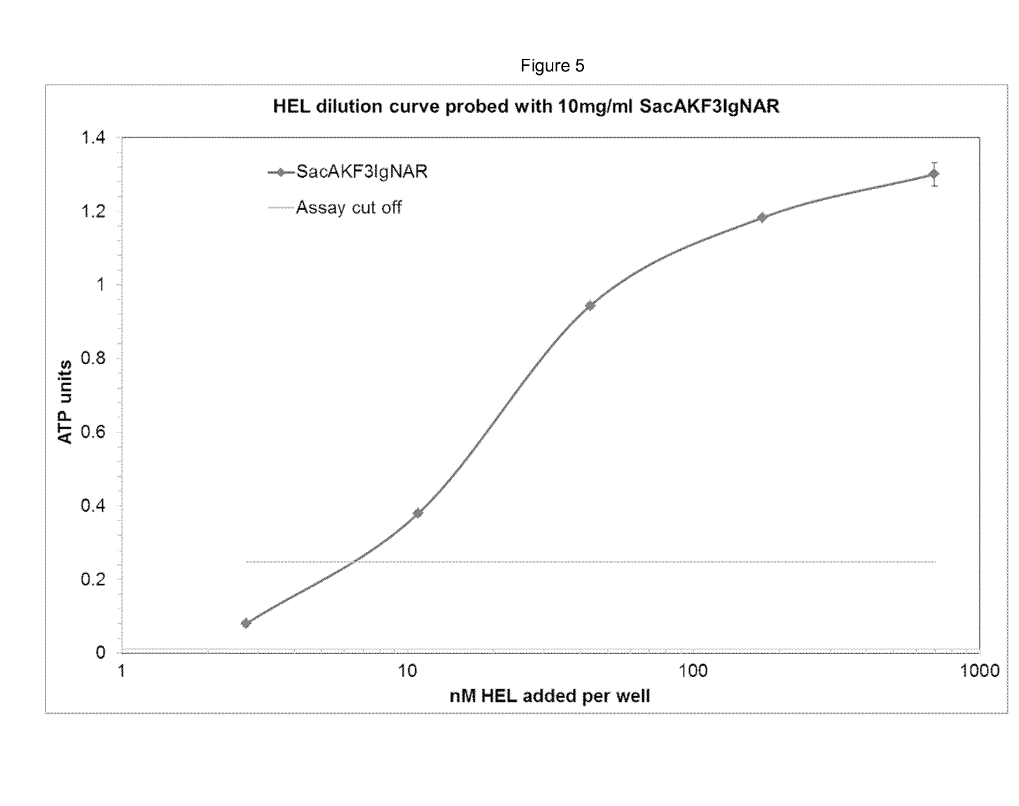

FIG. 5 illustrates detection of HEL by SacAK-F3-V_{NAR} at close to the estimated KD of the V_{NAR} at around 20 nM (Dooley et al.). Dilutions of HEL with final concentrations from 5-500 nM were generated in PBS, 100 ul was coated onto white Maxisorp plate and blocked with 5% BSA. The plate was probed with 10 μg/ml SacAK-F3-VNAR and the signal (corrected for background) was plotted. The level of sensitivity was defined as 3 SD above the blank (no antigen) control value. Results are typical of more than one experiment (n=6).

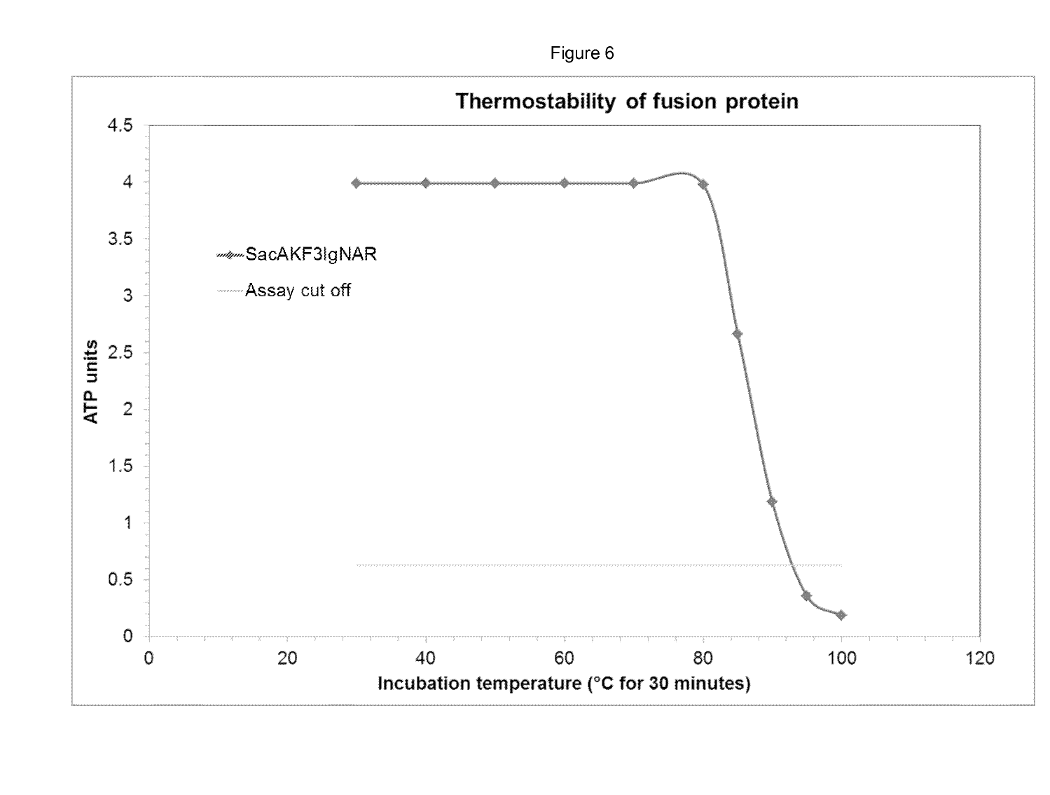

FIG. 6 illustrates retention of SacAK-F3-V_{NAR} binding and enzymatic activity after heat treatment at above 80° C. 10 μg/ml aliquots of SacAK-F3-V_{NAR} were incubated at a range of temperatures, cooled to room temperature then used to probe a plate coated with 10 μg/ml HEL. Activity of the fusion was unaffected up to 80° C., between 80-100° C. the signal dropped off significantly, with the melting temperature (temperature at which fusion retains half maximal activity) estimated at around 88.9° C. SacAK alone at equimolar concentrations and under the same salt concentrations, showed similar/different melting curves.

Similar results were observed with the Sac-F3-V_{H} fusion protein (results not shown) with activity of the Sac domain unaffected by temperatures up to 80° C. and approximately 10% of activity retained after 30 minutes treatment at 90° C.

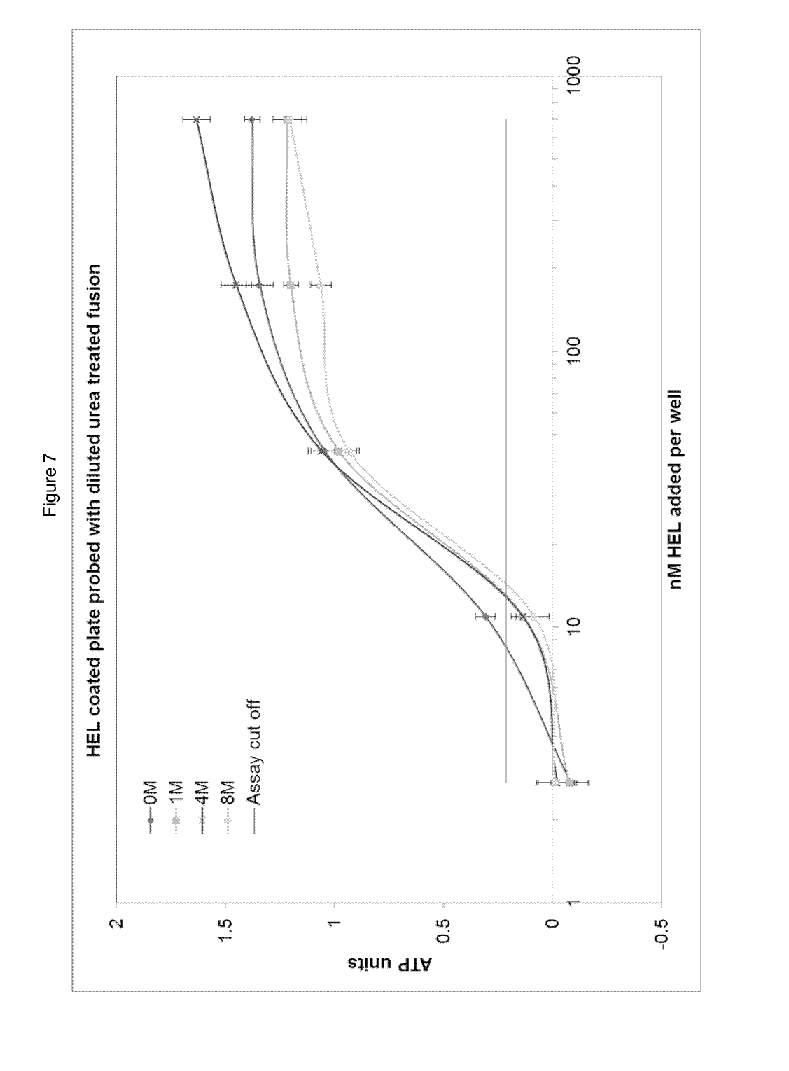

FIG. 7 illustrates that SacAK-F3-VNAR fusions retain activity after treatment with urea. 100 μg/ml of the fusion protein was treated with a final concentration of 0, 1, 4 or 8M urea for 30 minutes at 37° C. The sample was immediately diluted 1:10 and used to probe a Maxisorp plate coated with dilutions of HEL. The limits of detection of the assay, as defined by 3SD above the no antigen control value, remained unchanged under the conditions tested. Treatment of the bound HEL with the same concentration of urea reduced but did not abolish binding by the fusion (results not shown). Results are typical of more than one experiment (n=4).

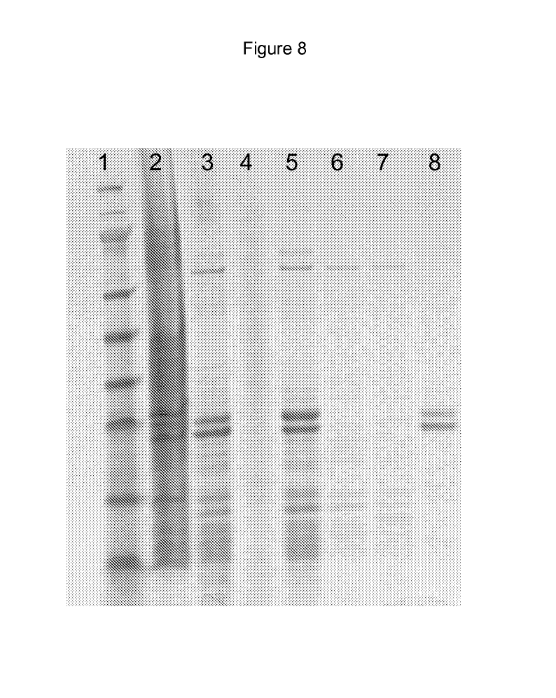

FIG. 8 illustrates SDS page analysis of purification of SacAK-F3-V_{H} fusion. Equal amounts of protein from each stage of the purification was loaded onto a 4-12% Bis Tris gel and electrophoresed in MES buffer under standard conditions. Purified SacAK-F3-VH is indicated with an arrow and lanes loaded as follows: 1: See Blue plus 2 ladder, 2: Insoluble homogenate, 3: Soluble homogenate, 4: Insoluble thermoprecipitate, 5: Soluble thermoprecipitate/column load, 6 Blue Sepharose column flow through 7: Blue Sepharose column wash, 8: Blue Sepharose column eluate. The Sac-F3-VH fusion runs consistently as a double band of approximate molecular weight 28 kDa consistent with observed migration of Sac fusion proteins on SDS-PAGE.

LIST OF SEQ ID NOs

SEQ ID NO 1: Acetate kinase from Methanosarcina
thermophila

| | |
|---|---|
| 1 | mkvlvinags sslkyglidm tnesalavgl cerigidnsi |
| | itqkkfdgkk lekltdlpth |
| 61 | kdaleevvka ltddefgvik dmgeinavgh rvvhggekft |
| | tsalydegve kaikdcfela |
| 121 | plhnppnmmg isacaeimpg tpmvivfdta fhqtmppyay |
| | myalpydlye khgvrkygfh |
| 181 | gtshkyvaer aalmlgkpae etkiitchlg ngssitaveg |
| | gksvetsmgf tpleglamgt |
| 241 | rcgsidpaiv pflmekeglt treidtlmnk ksgvlgvsgl |
| | sndfrdldea askgnrkael |
| 301 | aleifaykvk kfigeysavl ngadavvfta gigensasir |
| | kriltgldgi gikiddeknk |
| 361 | irgqeidist pdakvrvfvi ptneelaiar etkeivetev |
| | klrssipv |

SEQ ID NO 2: Pyruvate kinase from Geobacillus
stearothermophilus

| | |
|---|---|
| 1 | mkrktkivct igpasesvdk lvqlmeagmn varinfshgd |
| | heehgrrian ireaakrtgr |
| 61 | tvailldtkg peirthnmen gaielkegsk lvismsevlg |
| | tpekisvtyp sliddvsvga |
| 121 | killddglis levnavdkqa geivttvlng gvlknkkgvn |
| | vpgvkvnlpg itekdradil |
| 181 | fgirqgidfi aasfvrrasd vleirellea hdalhiqiia |
| | kieneegvan ideileaadg |
| 241 | lmvargdlgv eipaeevpli qkllikkcnm lgkpvitatq |
| | mldsmqrnpr ptraeasdva |
| 301 | naifdgtdav mlsgetaagq ypveavktmh gialrtegal |
| | ehrdilsqrt kesqttitda |
| 361 | igqsvahtal nldvaaivtp tvsgktpqmv akyrpkapii |
| | avtsneaysr rlalvwgvyt |
| 421 | keaphvnttd emldvavdaa vrsglvkhgd Ivvitagvpv |
| | getgstnlmk vhvisdllak |
| 481 | gqgigrksaf gkavvaktae earqkmvdgg ilvtvstdad |
| | mmpaiekaaa iiteegglts |
| 541 | haavvglslg ipvivgvena ttlfkdgqei tvdggfgavy |
| | rghasvl |

SEQ ID NO 3: UMP kinase, eg. from Pyrococcus
furiosus

| | |
|---|---|
| 1 | gsshhhhhhs sglvprgshm rivfdiggsv lvpenpdidf |
| | ikeiayqltk vsedhevavv |
| 61 | vgggklarky ievaekfnss etfkdfigiq itranamlli |
| | aalrekaypv vvedfweawk |
| 121 | avqlkkipvm ggthpghttd avaallaefl kadllvvitn |
| | vdgvytadpk kdptakkikk |
| 181 | mkpeelleiv gkgiekagss svidplaaki iarsgiktiv |
| | igkedakdlf rvikgdhngt |
| 241 | tiep |

SEQ ID NO 4: Butyrate kinase from Thermotoga
maritima

| | |
|---|---|
| 1 | mfriltinpg ststklsife dermvkmqnf shspdelgrf |
| | qkildqlefr ekiarqfvee |
| 61 | tgyslssfsa fvsrgglldp ipggvylvdg lmiktlksgk |
| | ngehasnlga iiahrfsset |
| 121 | gvpayvvdpv vvdemedvar vsghpnyqrk sifhalnqkt |
| | vakevarmmn kryeemnlvv |
| 181 | ahmgggisia ahrkgrvidv nnaldgdgpf tpersgtlpl |
| | tqlvdlcfsg kftyeemkkr |
| 241 | ivgngglvay lgtsdarevv rrikqgdewa krvyramayq |
| | iakwigkmaa vlkgevdfiv |
| 301 | ltgglaheke flvpwitkry sfiapvlvfp gsneekalal |
| | salrvlrgee kpknyseesr |
| 361 | rwrerydsyl dgilr |

SEQ ID NO 5: Adenylate kinase (monomeric) from
Thermotoga maritima

| | |
|---|---|
| 1 | mmaylvflgp pgagkgtyak rlgeitgiph istgdifrdi |
| | vkkendelgk kikeimerge |
| 61 | lvpdelvnev vkrrlsekdc ergfildgyp rtvaqaefld |
| | gflktqnkel taavlfevpe |
| 121 | evvvqrltar ricpkcgriy nlislppked elcddckvkl |
| | vgreddkeet vrhrykvyle |
| 181 | ktqpvidyyd kkgilkrvdg tigidnviae vlkiigwsdk |

SEQ ID NO 6: Adenylate kinase (trimeric) from
Sulfolobus acidocaldarius

| | |
|---|---|
| 1 | mkigivtgip gvgkstvlak vkeildnqgi nnkiinygdf |
| | mlatalklgy akdrdemrkl |
| 61 | svekqkklqi daakgiaeea raggegylfi dthavirtps |
| | gylpglpsyv iteinpsvif |
| 121 | lleadpkiil srqkrdttrn rndysdesvi letinfarya |
| | atasavlags tvkvivnveg |
| 181 | dpsiaaneii rsmk |

SEQ ID NO 7: Gene sequence for adenylate kinase
from Sulfolobus acidocaldarius with unstructured
linker and V_{NAR} sequence
ATGAAGATCGGCATTGTGACCGGCATTCCGGGCGTTGGCAAAAGCACCGTT
CTGGCAAAGGTGAAGGAGATCCTGGACAACCAGGGCATTAATAACAAAATT
ATTAATTATGGTGATTTTATGCTGGCGACCGCGCTGAAGCTGGGCTACGCA
AAAGATCGTGACGAAATGCGCAAACTGAGCGTGGAAAAACAGAAGAAGCTG
CAGATTGATGCGGCGAAGGGCATTGCGGAAGAGGCACGCGCGGGCGGCGAA
GGCTACCTGTTTATCGATACCCATGCGGTGATCCGCACCCCGAGCGGTTAT
CTGCCGGGCCTGCCGTCTTACGTGATTACGGAAATCAACCCGAGCGTTATT
TTTCTGCTGGAGGCAGATCCGAAGATTATTCTGAGCCGCCAGAAGCGCGAT
ACCACCCGCAACCGCAACGATTATAGCGACGAAAGCGTTATCCTGGAGACC
ATCAACTTTGCGCGCTATGCGGCAACCGCGAGCGCGGTTCTGGCAGGCTCT
ACCGTTAAAGTGATCGTGAACGTGGAGGGTGATCCAAGCATCGCGGCGAAC
GAAATCATTCGCAGCATGAAACAGTCGACCGGTGGTGGTGGTTCTGGTGGT
GGTGGTTCTGGTGGTGGTGGTTCTGGATCCGCACGTGTTGATCAGACACCG
CGTAGCGTTACCAAAGAAACCGGTGAAAGCCTGACCATTAATTGTGTTCTG
CGTGATGCAAGCTATGCACTGGGTAGCACCTGTTGGTATCGTAAAAAAAGC
GGCAGCACCAATGAAGAAAGCATTAGCAAAGGTGGTCGTTATGTTGAAACC
GTGAATAGCGGTAGCAAAAGCTTTAGCCTGCGTATTAATGATCTGACCGTT
GAAGATGGTGGGACCTACCGTTGTGGTCTGGGTGTTGCCGGTGGTTATTGT
GATTATGCACTGTGCAGCAGCCGTTATGCAGAATGTGGTGATGGTACTGCA
GTTACCGTGAAT
SEQ ID NO 8: V_{NAR} sequence for hen egg lysozyme
specific single domain antibody from shark.
GSARVDQTPRSVTKETGESLTINCVLRDASYALGSTCWYRKKSGSTNEES
ISKGGRYVETVNSGSKSFSLRINDLTVEDGGTYRCGLGVAGGYCDYALCS
SRYAECGDGTAVTVN
SEQ ID NO 9: Fusion protein sequence for adenylate
kinase from Sulfolobus acidocaldarius with
unstructured linker and V_{NAR} sequence
MLATALKLGYAKDRDEMRKLSVEKQKKLQIDAAKGIAEEARAGGEGYLFID
THAVIRTPSGYLPGLPSYVITEINPSVIFLLEADPKIILSRQKRDTTRNRN
DYSDESVILETINFARYAATASAVLAGSTVKVIVNVEGDPSIAANEIIRSM
KQSTGGGGSGGGGSGGGGSGSARVDQTPRSVTKETGESLTINCVLRDASYA
LGSTCWYRKKSGSTNEESISKGGRYVETVNSGSKSFSLRINDLTVEDGGTY
RCGLGVAGGYCDYALCSSRYAECGDGTAVTVN
SEQ ID NO 10: Fusion protein sequence for
adenylate kinase from Sulfolobus acidocaldarius
with helical linker and V_{NAR} sequence
MLATALKLGYAKDRDEMRKLSVEKQKKLQIDAAKGIAEEARAGGEGYLFID
THAVIRTPSGYLPGLPSYVITEINPSVIFLLEADPKIILSRQKRDTTRNRN
DYSDESVILETINFARYAATASAVLAGSTVKVIVNVEGDPSIAANEIIRSM
KQSTLAEAAAKEAAAKEAAAKEAAAKAAAGSARVDQTPRSVTKETGESLTI
NCVLRDASYALGSTCWYRKKSGSTNEESISKGGRYVETVNSGSKSFSLRIN
DLTVEDGGTYRCGLGVAGGYCDYALCSSRYAECGDGTAVTVN
SEQ ID NO 11: Fusion protein sequence for V_{NAR}
sequence with helical linker and adenylate kinase
from Sulfolobus acidocaldarius
MGSARVDQTPRSVTKETGESLTINCVLRDASYALGSTCWYRKKSGSTNEES
ISKGGRYVETVNSGSKSFSLRINDLTVEDGGTYRCGLGVAGGYCDYALCSS
RYAECGDGTAVTVNSTLAEAAAKEAAAKEAAAKEAAAKAAAGSMLATALKL
GYAKDRDEMRKLSVEKQKKLQIDAAKGIAEEARAGGEGYLFIDTHAVIRTP
SGYLPGLPSYVITEINPSVIFLLEADPKIILSRQKRDTTRNRNDYSDESVI
LETINFARYAATASAVLAGSTVKVIVNVEGDPSIAANEIIRSMK
SEQ ID NO 12: Fusion protein sequence for V_{NAR}
sequence with unstructured linker and adenylate
kinase from Sulfolobus acidocaldarius
MGSARVDQTPRSVTKETGESLTINCVLRDASYALGSTCWYRKKSGSTNEES
ISKGGRYVETVNSGSKSFSLRINDLTVEDGGTYRCGLGVAGGYCDYALCSS
RYAECGDGTAVTVNSTGGGGSGGGGSGGGGSGSMLATALKLGYAKDRDEMR
KLSVEKQKKLQIDAAKGIAEEARAGGEGYLFIDTHAVIRTPSGYLPGLPSY
VITEINPSVIFLLEADPKIILSRQKRDTTRNRNDYSDESVILETINFARYA
ATASAVLAGSTVKVIVNVEGDPSIAANEIIRSMK
SEQ ID NO 13: V_{H}H sequence for hen egg lysozyme
specific single domain antibody from camelid
EVQLQASGGGSVQAGQSLRLSCATSGATSSSNCMGWFRQAPGKEREGVAVI
DTGRGNTAYADSVQGRLTISLDNAKNTLYLQMNSLKPEDTAMYYCAADTST
WYRGYCGTNPNYFSYWGQGTQVTVSS
SEQ ID NO 14: Fusion protein sequence for
adenylate kinase from Sulfolobus
acidocaldarius with unstructured linker
and V_{H}H sequence
MLATALKLGYAKDRDEMRKLSVEKQKKLQIDAAKGIAEEARAGGEGYLFID
THAVIRTPSGYLPGLPSYVITEINPSVIFLLEADPKIILSRQKRDTTRNRN
DYSDESVILETINFARYAATASAVLAGSTVKVIVNVEGDPSIAANEIIRSM
KQSTGGGGSGGGGSGGGGSGSEVQLQASGGGSVQAGQSLRLSCATSGATSS
SNCMGWFRQAPGKEREGVAVIDTGRGNTAYADSVQGRLTISLDNAKNTLYL
QMNSLKPEDTAMYYCAADTSTWYRGYCGTNPNYFSYWGQGTQVTVSS
SEQ ID NO 15: Fusion protein sequence for
adenylate kinase from Sulfolobus acidocaldarius
with helical linker and V_{H}H sequence
MLATALKLGYAKDRDEMRKLSVEKQKKLQIDAAKGIAEEARAGGEGYLFID
THAVIRTPSGYLPGLPSYVITEINPSVIFLLEADPKIILSRQKRDTTRNRN
DYSDESVILETINFARYAATASAVLAGSTVKVIVNVEGDPSIAANEIIRSM
KQSTLAEAAAKEAAAKEAAAKEAAAKAAAGSEVQLQASGGGSVQAGQSLRL
SCATSGATSSSNCMGWFRQAPGKEREGVAVIDTGRGNTAYADSVQGRLTIS
LDNAKNTLYLQMNSLKPEDTAMYYCAADTSTWYRGYCGTNPNYFSYWGQGT
QVTVSS
SEQ ID NO 16: Fusion protein sequence for V_{H}H
sequence with helical linker and adenylate kinase
from Sulfolobus acidocaldarius
MEVQLQASGGGSVQAGQSLRLSCATSGATSSSNCMGWFRQAPGKEREGVAV
IDTGRGNTAYADSVQGRLTISLDNAKNTLYLQMNSLKPEDTAMYYCAADTS
TWYRGYCGTNPNYFSYWGQGTQVTVSSSTLAEAAAKEAAAKEAAAKEAAAK
AAAGSMLATALKLGYAKDRDEMRKLSVEKQKKLQIDAAKGIAEEARAGGEG
YLFIDTHAVIRTPSGYLPGLPSYVITEINPSVIFLLEADPKIILSRQKRDT
TRNRNDYSDESVILETINFARYAATASAVLAGSTVKVIVNVEGDPSIAANE
IIRSMK
SEQ ID NO 17: Fusion protein sequence for V_{H}H
sequence with unstructured linker and adenylate
kinase from Sulfolobus acidocaldarius
EVQLQASGGGSVQAGQSLRLSCATSGATSSSNCMGWFRQAPGKEREGVAVI
DTGRGNTAYADSVQGRLTISLDNAKNTLYLQMNSLKPEDTAMYYCAADTS
TWYRGYCGTNPNYFSYWGQGTQVTVSSSTGGGGSGGGGSGGGGSGSMLAT
ALKLGYAKDRDEMRKLSVEKQKKLQIDAAKGIAEEARAGGEGYLFIDTHA
VIRTPSGYLPGLPSYVITEINPSVIFLLEADPKIILSRQKRDTTRNRNDY
SDESVILETINFARYAATASAVLAGSTVKVIVNVEGDPSIAANEIIRSMK
SEQ ID NO: 18: Helical linker
LAEAAAKEAAAKEAAAKEAAAKAAA
SEQ ID NO: 19: Acetate kinase from
Thermotoga maritima
MRVLVINSGSSSIKYQLIEMEGEKVLCKGIAERIGIEGSRLVHRVGD
EKHVIERELPDHEEALKLILNTLVDEKLGVIKDLKEIDAVGHRVVHG
GERFKESVLVDEEVLKAIEEVSPLAPLHNPANLMGIKAAMKLLPGVP
NVAVFDTAFHQTIPQKAYLYAIPYEYYEKYKIRRYGFHGTSHRYVSK
RAAEILGKKLEELKIITCHIGNGASVAAVKYGKCVDTSMGFTPLEGL
VMGTRSGDLDPAIPFFIMEKEGISPQEMYDILNKKSGVYGLSKGFSS
DMRDIEEALKGDEWCKLVLEIYDYRIAKYIGAYAAAMNGVDAIVFTA
GVGENSPITREDVCSYLEFLGVKLDKQKNEETIRGKEGIISTPDSRV
KVLVVPTNEELMIARDTKEIVEKIGR
SEQ ID NO: 20: UMP kinase from Thermotoga
maritima.
MRVLVKLSGEALSGEGGRGFDPERVNYIVNEIKSAIEEGFKIGIVVG
AGNLFRGVELKNLTMTRADQIGLLGTVMNSVYLKDIFERSGLKARIY
SQIVNLPDVERVNYDSIESALRENSILIFAGGTSNPFFTTDTAAVLR
AQEMRAKLVVKATKVDGVYDKDPKKFPDAKKIPHLTFSEAMKMGLKV
MDAEAFALCKKLGITVKVINFFEPGTLLKALKGEDVGSTVVPD
SEQ ID NO: 21: UMP kinase from Sulfolobus
acidocaldarius.
MKLTLKVSGKFFDEENSENLSLLRDVIIDLVNNGHRVAVVTGGGGTA
RRYISMGRKLNLNESHLDILGILVSRLNAQLLLFSLDNIAYPKVPES
IEDFNERWASGKVVITGGFQPGQSTAGVAALVSEIINADYLVLATNV
NGVYTKDPQKFVDAKLLPKLTVSELKTILEGSQSVNAGKYELLDPLA
IKIVERSKIKVLVINFKDLNKLPNILKGNEILGSVVVPE

### EXAMPLES

### Example 1

### Cloning of IgNAR Gene Constructs

Sulfolobus acidocaldarius adenylate kinase (SacAK: GenBank: YP255258) coding sequence was synthesised using a high expressing E. coli codon bias and ligated into a pMTL1015 vector (GenBank: CS249842) using NdeI (R0111S) and SalI-HF (R3138S). A Gateway™ cassette (11828029, Invitrogen, Paisley, UK) was subsequently ligated into the XhoI (R0146S) site. The XhoI site had previously been blunted using T4 DNA polymerase (MO203S) prior to the ligation. The resulting cytoplasmic expression destination vector was called pTDCnSacAK-DEST. The destination vector was cloned into ccdB survival chemically competent cells (11828029, Invitrogen, Paisley, UK).

The HEL specific V_{NAR} ([E] GenBank: AAN75851) was synthesised with a high expressing E. coli codon bias and a BamHI site at the 5′ end then inserted into the commercially available pENTR/D-TOPO (Invitrogen). Cloning for the entry vector was carried out using chemically competent TOP10 cells (C404006, Invitrogen, Paisley, UK).

Recombination of the destination vector and entry vector was carried out using LR clonase II mix (11791020, Invitrogen, Paisley, UK) as per the manufacturer's instructions.

The gateway linker region, consisting of the gateway recombination site, was replaced with a flexible linker (F3) with the amino acid repeats [GGGGS]₃. The linker region was created as oligonucleotides (Sigma-Genosys, UK) with overlapping ends corresponding to the cleaved BamHI and SalI restriction sites. The oligonucleotides were annealed by incubating at 90° C. prior to insertion through traditional restriction digest of the vector and ligation.

Completed expression vectors were then transformed into E. coli strain RV308 and stored in cryoprotective beadstocks (PL170, Microbank, Prolab Diagnostics, Canada).

Synthesis and recombination of the constructs was confirmed by sequencing (Beckman Coulter Genomics, Takeley, UK) as was the insertion of alternative linker regions in the fusion protein.

Single domain antibody and/or single domain antibody fragment fusions may alternatively be rapidly generated due to the cassette-based method of construction used in assembling the fusion constructs (FIG. 1). Genetically engineered variants of SacAK or different thermostable kinases (e.g T. maritima acetate kinase or UMP kinase) can be inserted as NdeI-SalI fragments into the fusion construct to generate, for example, acetate kinase-F3 linker-single domain antibody fusions. Similarly, individual single domain antibody fragments or libraries of such fragments could be cloned into the fusions constructs as BamHI-XhoI fragments. This cassette approach circumvents the need for multiple cloning steps or any difficulties encountered with the recombination-based cloning systems (Gateway).

### Example 2

### Expression of IgNAR Fusion Proteins in E. coli

Expression was achieved using E. coli RV308 strain in Modified Terrific Broth (T0918). Primary seed (Luria-Bertani Broth+Tetracycline (T7660) 10 μg/ml) was inoculated with one colony from a Luria-Bertani Agar+Tetracyline (10 μg/ml) plate and left to grow overnight at 200 rpm and 37° C.

After overnight growth the primary seed was inoculated at 5 ml/litre into the Modified Terrific Broth flask culture (+10 μg/ml Tet); 200 rpm 37° C. with OD₆₀₀ monitored. When an OD₆₀₀>4 was achieved the cells were harvested by centrifugation at 3000 rpm for 30 minutes using a RC3BP (Sorvall, UK) centrifuge with H6000A rotor at 4° C.

The resulting cell paste was then resuspended in PBS and stored as aliquots at −80° C. until needed.

### Example 3

### Purification of Fusion Protein

Cell paste was homogenised to break the cells in a high salt buffer (20 mM Trizma-Base (T6066), 0.9M NaCl (S9888), 10 mM MgCl (H8266) pH8.0) with 2 mM Glutathione (G4251) added. The soluble fraction of the homogenate was then incubated at 80° C. for 20 minutes to precipitate out all non-thermostable proteins. Following centrifugation the soluble fraction was loaded onto a 5 ml Blue Sepharose FF Hitrap column (GE Healthcare, 17-0413-01) which was equilibrated in the same high salt buffer that was used to resuspend the cell paste for homogenisation. The glutathione was removed using a 1 hour gradient into 20 mM Trizma-base, 0.9M NaCl, 10 mM MgCL pH8.0. The fusion protein was then eluted using 20 mM Trizma-base, 0.9M NaCl, 10 mM MgCL, 10 mM ATP (A3377) and AMP (A1752) pH8.0. The column was run at 1 ml minute⁻¹, fractions were collected and analysed by SDS-PAGE gel and the fractions containing the final fusion protein product were pooled.

SDS-PAGE gels were run on NuPage 4-12% BisTris 15 well gels (NP0323BOX, Invitrogen, Paisley, UK) with NuPage MES SDS Running Buffer (20×) (NP0002, Invitrogen, Paisley, UK) with See Blue plus 2 ladder (LC5925, Invitrogen, Paisley, UK), 2.5 μg recombinant SacAK as on-gel controls. All samples were run in the presence of 5 μM DTT (D0632). Gels were developed using Simply Blue Safe Stain (LC6065, Invitrogen, Paisley, UK) and imaged using the Gel Doc.

### Example 4

### Characterisation of Fusion Protein Activity

White F96 MicroWell™ MaxiSorp plates (436110, Nunc, Denmark) were used in all assays.

All assays were read at the same gain setting (2553) on the BMG Labtech FLUOstar Omega plate reader.

In order to eliminate plate to plate variability each plate was also set up with an ATP dilution series from 5 down to 0.625 μMol per litre and this was used to convert the RLU to ATP units which are not subject to plate to plate variability.

### 4.1: Determination of AK Activity

Equimolar SacAK-F3-V_{NAR} fusion protein (50 ul) or the SacAK (50 μl) was added per well with 50 μl Luciferase reagent (Biothema ATP kit SL 144-04, Biothema AB, Sweden)+15 μM ADP (Sigma A1752, ATP depleted using 5 ml DEAE Sepharose FF HiTrap column (GE Healthcare, 17-5055-01)). The plate was incubated for 1 minute and luminescence read on a BMG Labtech FLUOstar Omega at 2553 gain as an endpoint reading after 15 seconds integration.

### 4.1: Detection of Antigen-Binding Activity of V_{NAR}

Functional ELISA was tested by coating the plate with a serial dilution of HEL (L6876) made up in PBS from 1 μg/well down with an additional BSA (A6003) control strip (50 μg/ml), for 1 hour shaken at 37° C. The plate was washed 3 times in PBS containing 1% Tween 20 (P1379) and then blocked in 5% BSA in PBS for 1 hour shaken at 37° C. The plate was washed 3 times in PBS containing 1% Tween 20. The fusion protein was bound to the plate at 10 μg/ml on both the HEL and BSA coated sides for 1 hour shaken at 37° C. and then washed in PBS containing Tween. Luciferase reagent (Biothema ATP kit SL 144-04, Biothema AB, Sweden) with the addition of 15 μM ADP (Sigma A1752, ATP depleted using 5 ml DEAE Sepharose FF HiTrap column (GE Healthcare, 17-5055-01)) was added to the plate. The plate was incubated for 1 minute and luminescence read as Relative Luminescence Units (RLU) on a BMG Labtech FLUOstar Omega at 2553 gain as an endpoint reading after 15 seconds integration.

### Example 5

### Demonstration of Stability

### 5.1 Thermostability Studies

A plate was coated with 100 ul/well of 10 μg/ml HEL (L6876) as well as a BSA (A6003) control for 1 hour shaken at 37° C. The plate was washed 3× in PBS+Tween 20 (P1379) and then blocked in 5% BSA in PBS for 1 hour shaken at 37° C. The plate was washed 3× in PBS+Tween 20.

Purified, characterised fusion (10 μg/ml) was incubated in separate tubes for 30 minutes at a range of temperatures up to 100° C.

Aliquots (100 μl/well) of the thermally treated fusion were then applied to the plate and incubated for 1 hour, shaken at 37° C. The plate was washed 3× in PBS+Tween 20.

The plate was developed and read as per the functional ELISA described above.

### 5.2 Denaturant Stability Studies

Denaturant studies were performed as per the functional ELISA above. To test the stability of the SacAK-F3-V_{NAR}, aliquots (200 μg/ml) were treated with a range of urea concentrations up to 8M urea for 1 hour at room temperature then diluted to 10 μg/ml (giving a maximum urea concentration of 0.4M). The plate was then probed with these aliquots to prevent the bound HEL from being denatured.

### Example 6

### Cloning of V_{H}H Gene Constructs

Generation of the Sulfolobus acidocaldarius adenylate kinase (SacAK: GenBank: YP255258) construct was as described in Example 1.

Similar approaches were used for the other thermostable kinases as described herein (SEQ ID NOs 1-7).

In brief, the thermostable kinases genes were re-synthesised with E. coli codon bias or, if the codon usage was acceptable, the genes of interest were PCR amplified from genomic DNA. Gene constructs were sequence verified and sub-cloned into the destination vector, as detailed in Example 1. Final expression constructs were also sequence verified.

The HEL specific V_{H}H fragment (GenBank: 1RJC_Acession 29 Oct. 2010) was synthesised with a high expressing E. coli codon bias and a BamHI site at the 5′ end, and then inserted into the commercially available pENTR/D-TOPO (Invitrogen). Cloning for the entry vector was carried out using chemically competent TOP10 cells (C404006, Invitrogen, Paisley, UK).

Recombination of the destination vector and entry vector was carried out using LR clonase II mix (11791020, Invitrogen, Paisley, UK) as per the manufacturer's instructions.

The gateway linker region, consisting of the gateway recombination site, was replaced with a flexible linker (F3) with the amino acid repeats [GGGGS]₃. The linker region was created as oligonucleotides (Sigma-Genosys, UK) with overlapping ends corresponding to the cleaved BamHI and SalI restriction sites. The oligonucleotides were annealed by incubating at 90° C. prior to insertion through traditional restriction digest of the vector and ligation.

Completed expression vectors were then transformed into E. coli strain BL21, BLR, RV308 or Origami and stored in cryoprotective beadstocks (PL170, Microbank, Prolab Diagnostics, Canada).

Synthesis and recombination of the constructs was confirmed by sequencing (Beckman Coulter Genomics, Takeley, UK) as was the insertion of alternative linker regions in the fusion protein.

### Example 7

### Expression of V_{H}H Fusion Proteins in E. coli

Expression was achieved using E. coli RV308 strain in Modified Terrific Broth (T0918) or alternative media (Luria-Bertani—Broth or minimal medium). Primary seed (Luria-Bertani Broth+Tetracycline (T7660) 10 μg/ml) was inoculated with one colony from a Luria-Bertani Agar+Tetracyline (10 μg/ml) plate and left to grow overnight at 200 rpm and 37° C. Expression from other E. coli strains had limited effect on the expression levels produced and subsequent down-stream purification.

After overnight growth the primary seed was inoculated at 5 ml/litre into the Modified Terrific Broth flask culture (+10 μg/ml Tet); 200 rpm 37° C. with OD₆₀₀ monitored. When an OD₆₀₀>4 was achieved the cells were harvested by centrifugation at 3000 rpm for 30 minutes using a RC3BP (Sorvall, UK) centrifuge with H6000A rotor at 4° C.

The resulting cell paste was then resuspended in PBS and stored as aliquots at −80° C. until needed.

### Example 8

### Purification of Sac-F3-VH Fusion Protein

Camelid fusion proteins were purified essentially as described above (Example 3). Specifically:

Cell paste was lysed by sonication to break the cells in a high salt buffer (20 mM Trizma-Base, 0.9M NaCl, 10 mM MgCl pH8.0) with 2 mM Glutathione G4251) added. The soluble fraction of the homogenate was then incubated at 80° C. for 30 minutes to precipitate out all non-thermostable proteins. Insoluble proteins were removed by centrifugation and the soluble fraction was loaded onto a 5 ml Blue Sepharose FF Hitrap column. The glutathione was removed using a 1 hour gradient into the same buffer lacking glutathione. The fusion protein was then eluted using 20 mM Trizma-base, 0.9M NaCl, 10 mM MgCL, 10 mM ATP (A3377) and AMP (A1752) pH8.0. The column was run at 1 ml minute⁻¹, fractions were collected and analysed by SDS-PAGE gel and the fractions containing the final fusion protein product were pooled.

SDS-PAGE gels were run on NuPage 4-12% BisTris 12 well gels with NuPage MES SDS Running Buffer. Gels were developed using Simply Blue Safe Stain.

### Example 9

### Detection Reagents for Viral Pathogens; e.g. Ebola

Detection reagents are generated essentially as described in Examples 1-3 (shark antibody) or Examples 6-8 (camelid antibody). The single domain antibody is cloned as a BamHI-XhoI fragment into an expression vector, such as the pMTL expression vector described above, downstream of the Sac adenylate kinase gene.

Suitable single domain antibody fragments such as the ebola specific shark Ig_{NAR} domains (“Isolation and characterisation of Ebolavirus-specific recombinant antibody fragments from murine and shark immune libraries. 2011 Goodchild S A, Dooley H, Schoepp R J, Flajnik M, Lonsdale S G. Mol. Immunol. 48:2027-37) may be used to generate specific reagents.

Expression and purification of the sac-F3-IgNAR reagents may be achieved as above. Thermal stability and specificity for the target antigen would be determined essentially as outlined in Examples 4 and 5.

The reagent could be used in a standard detection assay, such as an ELISA. In brief, a capture antibody would be coated onto a solid support and remaining binding sites blocked by addition of a blocking agent such as 5% skimmed milk (3% BSA, 5% gelatin, or 0.1% Tween 20 could be used either in conjunction with the skimmed milk or in its place). A suitable clinical sample, in this case clarified whole blood or serum, would be applied to the capture-antibody-coated solid support and the target antigen (virus) allowed to bind. Unbound material would be removed by washing and/or selective removal of the solid support from the applied clinical sample.

The reagent can also be used in a magnetic bead-based solid support assay. Essentially the assay is performed as described above except that the capture antibody is coated onto a magnetic bead. The antigen capture and detection reactions are then carried out on beads which may be removed from the clinical sample matrix by attraction to a magnet. Rapid washing may also be achieved by mixing the beads with a saline solution, e.g. phosphate buffered saline containing 0.1% Tween 20.

### Example 10

### Detection Reagents for Bacterial Pathogens

Detection reagents are generated essentially as described in Examples 6-8. A single domain antibody fragment is suitable, such as those derived from library screens of camelid antibodies (e.g “Rugged single domain antibody detection elements for Bacillus anthracis spores and vegetative cells. Walper S A, Anderson G P, Brozozog Lee P A, Glaven R H, Liu J L, Bernstein R D, Zabetakis D, Johnson L, Czarnecki J M, Goldman E R. PLoS One. 2012; 7(3):e32801. Epub 2012 Mar. 6.” Or “Evaluation of a nanobody phage display library constructed from a Brucella-immunised camel. Abbady A Q, Al-Marin A, Zarkawi M, Al-Assad A, Muyldermans S. Vet Immunol Immunopathol. 2011 Jul. 15; 142(1-2):49-56. Epub 2011 Apr. 14”). The antibody is fused at the C-terminus of the Thermotoga maritima acetate kinase to generate the fusion protein Tma Acetate kinase-F3 linker-V_{H}. The fusion protein is expressed in RV308 or BL21 E. coli strain and the enzyme purified by heat treatment of cell lysate at 80° C. for 30 minutes. Optionally the fusion protein may be further purified by binding to a dye-ligand column (e.g. Blue sepharose).

The reagent can then be used in a solid phase binding assay in either an ELISA format or on magnetic beads, essentially as described in example 9. The acetate kinase activity may be detected in a reaction to generate ATP, using highly purified ADP (up to 10 mM) and acetyl phosphate (up to 100 mM). ATP can be detected by a variety of means including bioluminescent detection using luciferin/luciferase.

A similar approach may be taken to detect the product of a bacterial pathogen, especially protein toxins that may be closely linked with the pathogenesis. Examples of antibodies that may be generated and used in such constructs include “Isolation of a highly thermal stable lama single domain antibody specific for Staphylococcus aureus enterotoxin B. Graef R R, Anderson G P, Doyle K A, Zabetakis D, Sutton F N, Liu J L, Serrano-González J, Goldman E R, Cooper L A. BMC Biotechnol. 2011 Sep. 21; 11:86” and “Camelid single domain antibodies (VHHs) as neuronal cell intrabody binding agents and inhibitors of Clostridium botulinum neurotoxin (BoNT) proteases. Tremblay J M, Kuo C L, Abeijon C, Sepulveda J, Oyler G, Hu X, Jin M M, Shoemaker C B. Toxicon. 2010 November; 56(6):990-8. Epub 2010 Jul. 14” and “Isolation and Characterization of Clostridium difficile Toxin-Specific Single-Domain Antibodies. Hussack G, Arbabi-Ghahroudi M, Mackenzie C R, Tanha J. Methods Mol. Biol. 2012; 911:211-39”.

### Example 11

### Detection and Therapeutic Agents for Metalloprotease

A significant advantage of single domain antibodies, such as those from shark and camelid is their ability to bind the active site of enzymes, characterised by their deep hydrophobic cleft. Antibodies may be selected for this purpose from single domain antibody libraries, in phage display vectors or equivalent, by altering the binding and elution conditions to favour hydrophobically bound antibodies and/or those eluted by substrate competition. An example of such antibodies, suitable for this purpose is described in “Molecular imprint of enzyme active site by camel nanobodies: rapid and efficient approach to produce abzymes with alliinase activity. Li J W, Xia L, Su Y, Liu H, Xia X, Lu Q, Yang C, Reheman K. J Biol. Chem. 2012 Apr. 20; 287(17):13713-21. Epub 2012 Feb. 28”.

Such single domain antibodies may be expressed as a fusion with one or more of the thermostable kinases identified herein, such as the uridylate (UMP) kinase from Thermotoga maritima. The fusion is constructed such that the antibody domain is on the N-terminus of the kinase to generate a fusion protein, IgNAR-helical linker-Tma UMP kinase. The reagent is cloned, expressed and purified essentially as described in Examples 1-3, with a high temperature purification step (80° C. for 30 minutes) followed by an affinity capture step on Blue Sepharose. Other dye-ligand columns may also optionally be used to purify the fusion protein.

The reagent can be used in a detection or diagnostic assay, e.g. for the New Delhi metalloprotease associated with high level antibiotic resistance to beta-lactamase antibiotics. The assay may be formatted in a similar way to the solid phase binding assays described above using a separate capture antibody to enrich the target from a clinical sample or culture of bacterial pathogen. The UMP-kinase activity may be measured in a reaction that generate ATP, involving the co-substrates ADP (up to 100 mM) and UDP (up to 100 mM) at near neutral pH. The ATP generated may be detected by a number of means, including bioluminescent detection with luciferin-luciferase.

### Example 12

### Multivalent Display of Single Domain Antibodies on Multimeric Thermostable Kinases

The multimeric display of single domain antibodies may be achieved by co-expressing two or more fusion constructs in the same E. coli cell. Preferentially a recombination-defective E. coli strain is used to minimise any chance of re-arrangement of the gene constructs during expression. The two fusion protein constructs may be expressed from the same plasmid using the same or different promoters. Alternatively two different compatible plasmids may be used with one gene construct expressed from each. The ratio of different fusion proteins may be varied by using high or low copy number plasmids and/or high, medium or low expressing promoters. Any expressed multimeric thermostable kinase will have a variety of different multimeric-antibody species.

Two constructs are generated such that different single domain antibodies are attached to the thermostable UMP-kinase from Thermotoga maritima, enabling hexameric display of the single domain antibodies. The two constructs are expressed from a single pMTL vector essentially as described in Example 1, with each gene construct driven by its own promoter. The constructs are generated such that the fusions are Tma UMP-kinase-F3 linker-IgNAR1 and Tma UMP-kinase-helical linker-IgNAR2. The constructs are expressed at similar levels in E. coli RV308 (recombinase-negative) and can be expressed and purified essentially as described in Examples 1-3 above. The population of antibody-displaying hexameric kinases will include constructs expressing different ratios of IgNAR1 to IgNAR2 fusions. Optionally constructs may be further purified using an affinity column loaded with the ligands for each of the IgNAR domain to ensure that all hexamers contain at least one of each antibody domain.

A specific example would be the hexameric display of single domain antibodies recognising different epitopes on both C. difficile toxin A and toxin B, such that the constructs are Tma UMP-kinase-F3 linker-IgNAR; toxin A and Tma UMP-kinase-F3 linker-IgNAR; toxin B. The equivalent camelid domains such as those described in the Hussack paper (Example 10) would also be valuable for developing this type of reagent.

Similar constructs can be generated with any of the other multimeric thermostable kinases described herein, such that di-, tri-, tetra-, penta-, hexa-, or octa-valent display of single domain antibodies can be achieved.

### Example 13

### Applications of Mosaic (Multivalent) Antibody Display Reagents in Detection and Diagnostics

### i) Simple Capture Assays Using Mosaic Reagents.

The multimeric, “mosaic” antibody-thermostable kinase detection reagents may be used in any of the different assay formats such as those described in the examples above, e.g. for detection of bacteria, toxins, viral pathogens, specific enzymes or other analytes. The avidity effect caused by having multiple copies of one or more antibody domain make them extremely useful antibody detection reagents for high sensitivity and/or rapid detection systems. The ability to co-express the antibody reagent and the detection enzyme (thermostable kinase) also make them simple and easy to use for rapid generation of new reagents.

Preferred applications are those where the different single domain antibodies allow the detection of target species, using a single reagent, where there is significant antigenic diversity that might not allow the use of a single monoclonal antibody. For example, viral pathogens such as norovirus and influenza have the ability to rapidly shift antigen diversity and evade monoclonal antibody binding. By having two or more antibodies recognising different antigens, this risk is minimised. Similarly diseases where two different toxins, either individually or in combination, can mediate disease may be beneficially detected using the reagents described here. This is the case for Clostridium difficile where toxin B either with or without toxin A can mediate disease; both toxins being recognised as risk factors. Many other examples of the benefits of multivalent detection reagents are known to those familiar with the art and this is often cited as one of the benefits of using polyclonal antibody reagents. These are however, slow to make, tend to be variable in quality and do not have the benefits of the highly-defined, enzyme-tagged polyvalent reagents described here.

### A Specific Example of the Use of Such an Assay:

A polyvalent Tma UMP-kinase was generated as described in Example 12, with two different Ig_{NAR} domains recognising epitopes on C-difficile toxin A and B, respectively. Stool extracts from suspected clinical patients were processed using standard methods and applied to an ELISA assay coated with capture reagents (polyclonal antibodies) recognising both C. difficile toxins A and B. The plates were incubated and unbound material removed by washing. The UMP-kinase detection reagent was added and the plate incubated at 37° C. for 30 minutes to allow binding of the Ig_{NAR} domains to the captured toxin molecules. The plate was again washed to remove unbound detection reagent and the UMP-kinase activity measured by simultaneous addition of ADP, UDP luciferin and luciferase in magnesium acetate buffer. The assay can be used for rapid detection of clinical isolates expressing toxin B and toxins A and B. The assay would also detect toxin A only clinical isolates.

A further example would be use of a mosaic detection reagent recognising norovirus genogroups GI and GII. Multivalent reagents are generated by expressing separate single domain antibodies, specific for GI and GII respectively, fused to the trimeric thermostable adenylate kinase from Sulfolobus acidocaldarius. Expression and purification of the reagents and subsequent use in the assay are as described above.

### ii) Recruitment of Additional Detection Reagents.

To increase the sensitivity of the detection reaction, one of the fusion protein generated is specific for an additional thermostable kinase. For example, in an assay to detect anthrax spores, multivalent detection reagents are generated using two constructs; e.g. Tma UMP-kinase-F3-linker-anthrax spore specific V_{H} domain, and Tma UMP-kinase-F3-linker-single domain antibody for Tma acetate kinase. The genes are expressed from the same plasmid but using different regulated promoters such that the fusion proteins are expressed in a ratio of 1 anthrax spore specific fusion to 2 acetate kinase specific fusion. This gives rise to a distribution of hexameric UMP-kinase fusion with a bias towards 2 anthrax spore specific single domain antibodies and 4 acetate kinase single domain antibodies. When used in an assay, using substrates ADP, UDP, acetyl phosphate, followed by luciferin and luciferase, this results in up to 8-fold greater signal intensity compared to the UMP kinase reagent alone (based on the recruitment of 4 additional acetate kinase dimers capable of catalysing the production of ATP).

A similar approach may be used to generate different types of diagnostic signal. For example the co-expression of (i) a fusion protein comprising a trimeric Sac-adenylate kinase fusion with (ii) an antibody for a chlamydial antigen together with an antibody specific for a fluorophore allows for rapid detection in a standard ATP-generating assay, as described above, as well as staining of tissue sections to determine the intracellular fate of the pathogen (i.e. indicating whether infection has been established). A similar approach could be used with a single domain antibody for a tracer molecule which could be imaged in a whole body system, or via histology staining.

1-17. (canceled)

18. A single-chain fusion protein comprising:
(i) a multimeric thermostable kinase and
(ii) a single-domain antibody or single-domain antibody fragment.

19. The fusion protein according to claim 18, wherein the multimeric thermostable kinase is a trimeric, tetrameric, hexameric or octameric thermostable kinase.

20. The fusion protein according to claim 18, wherein the multimeric thermostable kinase is an adenylate kinase, acetate kinase, uridine monophosphate (UMP) kinase, pyruvate kinase, or butyrate kinase.

21. The fusion protein according to claim 18, wherein the multimeric thermostable kinase is a trimeric adenylate kinase.

22. The fusion protein according to claim 18, wherein the multimeric thermostable kinase is from a Sulfolobus species, such as S. acidocaldarius.

23. The fusion protein according to claim 18, wherein the multimeric thermostable kinase is a trimeric adenylate kinase from S. acidocaldarius.

24. The fusion protein according to claim 18, wherein the single-domain antibody is a shark new antigen receptor (IgNAR) or fragment thereof; or wherein the single-domain antibody is a camelid antibody or fragment thereof.

25. (canceled)

26. (canceled)

27. (canceled)

28. A method of preparing a single-domain antibody or single-domain antibody fragment, the method comprising:
(i) expressing the single-domain antibody or antibody fragment as a single-chain fusion protein with a multimeric thermostable kinase, in a host cell; and
(ii) purifying the fusion protein from the cytoplasm of the host cell.

29. The method according to claim 28, wherein the fusion protein is as defined in claim 18.

30. A multimeric fusion protein complex comprising two or more fusion proteins according claim 18, wherein the thermostable kinase is a multimeric thermostable kinase.

31. The multimeric fusion protein complex according to claim 30, wherein the single-domain antibodies or antibody fragments fused to the thermostable kinase monomers of the two or more fusion proteins are the same.

32. A reporter molecule for detecting an analyte on or within a sample, the reporter molecule comprising a fusion protein according to claim 18.

33. A detection kit comprising:
(i) a fusion protein according to any claim 18, a multimeric fusion protein complex according to claim 30, or a reporter molecule according to claim 32, and
(ii) a substrate for the multimeric thermostable kinase.

34. An in vitro method of detecting an analyte on or within a sample, comprising:
(i) contacting the sample with a reporter molecule according to claim 32, wherein the single-domain antibody or antibody fragment binds the analyte to form an antibody-analyte complex; and
(ii) detecting the antibody-analyte complex by detecting the kinase activity of the thermostable kinase.
